(19) United States
(12) Patent Application Publication
(10) Pub. No.: US 2025/0066411 A1
(43) Pub. Date: Feb. 27, 2025

(54) GLYCOMIMETIC BINDERS FOR L-SIGN

(71) Applicants: UNIVERSITA’ DEGLI STUDI DI MILANO, Milano (IT); UNIVERSITÉ GRENOBLE ALPES, Saint Martin d’Hères (FR); COMMISSARIAT A L’ÉNERGIE ATOMIQUE ET AUX ÉNERGIES ALTERNATIVES, Paris (FR); CENTRE NATIONAL DE LA RECHERCHE SCIENTIFIQUE, Paris (FR)

(72) Inventors: Anna BERNARDI, Milano (IT); Sara POLLASTRI, Milano (IT); Clara DELAUNAY, Grenoble (FR); Michel THÉPAUT, La Buisse (FR); Franck FIESCHI, Varces Allières Et Risset (FR); Gianluca CAVAZZOLI, Milano (IT)

(73) Assignees: UNIVERSITA’ DEGLI STUDI DI MILANO, Milano (IT); UNIVERSITÉ GRENOBLE ALPES, Saint Martin d’Hères (FR); COMMISSARIAT A L’ÉNERGIE ATOMIQUE ET AUX ÉNERGIES ALTERNATIVES, Paris (FR); CENTRE NATIONAL DE LA RECHERCHE SCIENTIFIQUE, Paris (FR)

(21) Appl. No.: 18/925,568

(22) Filed: Oct. 24, 2024

Related U.S. Application Data
(63) Continuation-in-part of application No. PCT/IB2023/054291, filed on Apr. 26, 2023.

(30) Foreign Application Priority Data
Apr. 28, 2022 (IT) 10202200008474
Apr. 28, 2022 (IT) 102022000008474

(51) Int. Cl. C07H 19/056; A61K 31/7056; A61K 31/7064

(52) U.S. Cl. CPC C07H 19/056; A61K 31/7056; A61K 31/7064

(57) ABSTRACT

The present invention relates to new glycomimetic molecules which selectively bind to the L-SIGN receptor, finding application in the medical field and, in particular, in the prevention and treatment of viral infections, in the immunotherapy of liver tumors and in the treatment of immune diseases.

### FIELD OF THE INVENTION

The present invention relates to selective glycomimetic ligands for the L-SIGN receptor, which find application in the medical field and, in particular, in the prevention and treatment of viral infections, for example caused by SARS-CoV-2, in the immunotherapy of liver tumors and in the treatment of immune diseases.

### State of the Art

The SARS-CoV-2 virus infects human cells by exploiting the interaction between the membrane protein ACE2 (angiotensin-converting enzyme 2) and Spike glycoprotein.

The SARS-CoV-2 virus also employ some immune cells to increase its transmission capacity. More specifically, the Spike glycoprotein is able to interact with human receptors belonging to the lectin family, called “C-type lectin receptors” (CLRs), including DC-SIGN and L-SIGN. The CLRs receptors are located on the surface of immune cells and are widely present in the respiratory mucosa and lung tissue.

The capture of the virus by the CLRs receptors does not induce direct cellular infection; however, DC-SIGN and L-SIGN are able to retain the virus and transmit it to the permissive cells on which the ACE2 membrane protein is present, promoting the trans-infection of the virus itself.

The virus transmission processes mediated by CLR receptors can be inhibited by using molecules that mimic the glycans present on the surface of the virus (glycomimetics).

The DC-SIGN receptor is expressed in immature dendritic cells. In contrast, the L-SIGN receptor is expressed in type II alveolar cells and endothelial cells of the human lung and liver, and is co-expressed with ACE2 in respiratory tract cells.

Therefore, in therapies to counter SARS-CoV-2 infection, L-SIGN is a more relevant target than DC-SIGN. The same is true for numerous other antiviral therapies. For example, L-SIGN is known to be the preferred attachment factor for West Nile virus (Davis et al J. Virol. 2006, 1290).

To date, there are no selective glycomimetic molecules for the L-SIGN receptor with respect to DC-SIGN. Therefore, the problem behind the present invention is to develop glycomimetic molecules that selectively bind to L-SIGN and do not interact effectively with DC-SIGN.

### SUMMARY OF THE INVENTION

The problem set out above is solved by a new class of glycomimetic molecules, as outlined in the attached claims, the definitions of which forms an integral part of the present description.

Surprisingly, it has been found that these molecules, despite being closely related to a chemotype that contains active DC-SIGN ligands, possess a high selectivity for the L-SIGN receptor. Thus, they can be effective in the prevention and treatment of infections by viruses that preferentially bind to the L-SIGN receptor. They can also be employed as targeting agents of drugs and vaccines towards liver endothelial cells (LSECs) expressing the L-SIGN receptor.

Further characteristics and advantages of the invention will become clearer in the description of some embodiment examples, given hereinafter by way of indication and not of limitation.

### BRIEF DESCRIPTION OF THE FIGURES

FIG. 1 illustrates the results of the competition experiments performed by Surface Plasmon Resonance for the glycomimetic ligands identified in the experimental part of the present application as compounds 106a, 113, 115, 119, 139 and 146 (in accordance with the invention), the molecule identified in the experimental part as compound 103 and mannose (comparison examples).

FIG. 2 illustrates the data concerning the Isothermal Titration Calorimetry experiments carried out on the glycomimetic ligand identified in the experimental part of the present application as compound 106a (in accordance with the invention);

FIG. 3 shows the results of the direct interaction experiments performed by Surface Plasmon Resonance for the ligands identified in the experimental part of the present application as compounds 133 and 135 (in accordance with the invention).

### DETAILED DESCRIPTION OF THE INVENTION

The object of the present invention is a compound of formula (I) or a salt thereof,

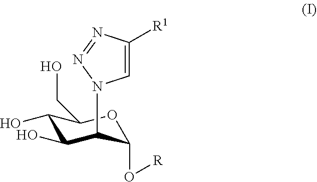

in which R is selected from the group consisting of: C₁-C₆ alkyls, C₃-C₆ cycloalkyls, C₆-C₁₀ aryls, —R²Cl, —R³NH₂, —R⁴NHC(O)R′, —R⁵SR″, —R⁶OR″, and

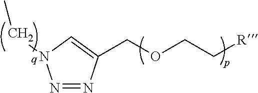

where:
p is an integer from 0 to 3;
q is an integer from 1 to 3;
R², R³, R⁴, R⁵, R⁶ are divalent radicals selected from the group consisting of C₁-C₆ alkylenes and C₃-C₆ cycloalkylenes, preferably C₂ alkylenes having formula —CH₂CH₂—, and
R′, R″ are selected from the group consisting of C₁-C₆ alkyls, C₃-C₆ cycloalkyls and C₆-C₁₀ aryls,
R′″ is selected from the group consisting of —OH and halogens, preferably Cl,
and in which R¹ has formula (II):

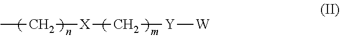

where:
X is selected from the group consisting of CH₂, O, NH and S;
Y is selected from the group consisting of O, NH and S;
n is an integer between 0 and 3;
m is an integer from 0 to 3, preferably 0;
W is a heterocyclic substituent W1 selected from the group consisting of:

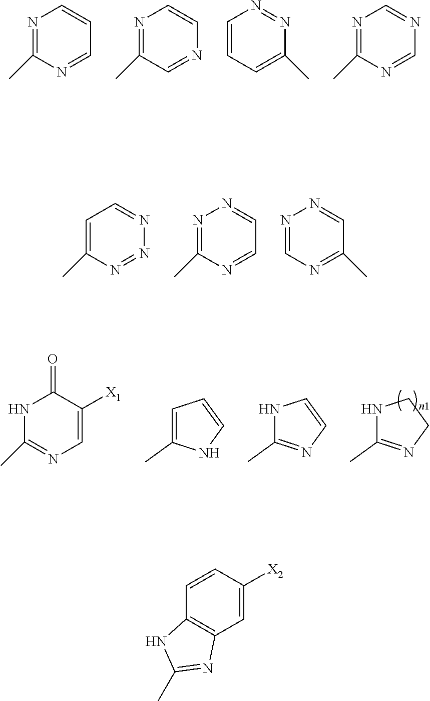

or it is a substituent W2 having general formula:

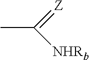

where:
X₁ is selected from the group consisting of H and C₁-C₆ alkyls preferably H or methyl;
X₂ is selected from the group consisting of H, OCH₃, OH,
NH₂, CH₃, CF₃, SH, SR, halogen, where R is as defined above preferably R being selected from C₁-C₆ alkyls;
n₁ is an integer selected between 1 and 2;
Z is selected from the group consisting of S and NRₐ,
in which Rₐ and R_{b} are independently selected from the group consisting of H, COR⁷, COOR⁹, CONHR⁹, where R⁷, R⁸ and R⁹ are H, C₁-C₆ alkyls, C₃-C₆ cycloalkyls or C₆-C₁₀ aryls.

The present invention also relates to a compound of formula (IA) or a salt thereof,

A-(B)-C-(B)-A (IA)
in which:

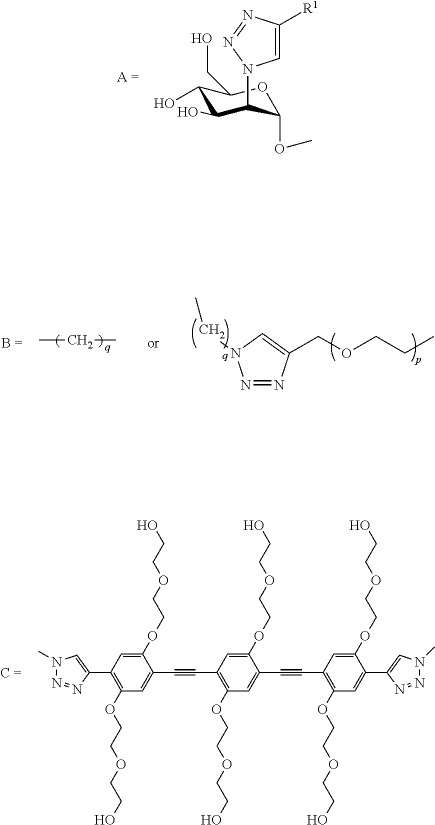

where R¹, p and q are as defined above.

Said compound of formula (IA) is a dimer of the above compound of formula (I).

Preferably, the R group of the compound of formula (I) is —R²Cl, more preferably —CH₂CH₂Cl.

According to a first embodiment, the group R¹ has formula (II-A)

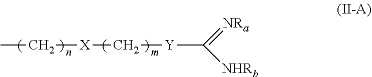

wherein when n=0, X is CH₂.

In accordance with this embodiment, preferably, X is CH₂ and Y is selected from NH, S and O. Preferably, n=0 and/or m=0. Preferably, Rₐ═H and/or R_{b}═H.

In accordance with another embodiment, preferred compounds of formula (I) are those compounds wherein:
the substituent R¹ has the formula (II-A) wherein n and m are 0, X is CH₂, Y is NH and Ra═Rb=H or Ra and Rb form, together with the heteroatoms to which they linked, an imidazole or imidazoline or benzimidazole or tetrahydropyrimidine moiety, that can be unsubstituted or substituted with a substituent preferably selected from alkyl, alkoxy, halogen, cycloalkyl, cycloalkoxy, aryl and heteroaryl;
the substituent R is a group —CH₂CH₂Cl.

In accordance with this embodiment, the compound of formula (I) has the following formula:

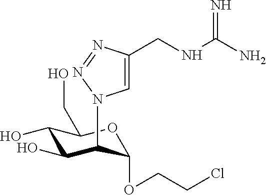

or it is a salt thereof, preferably a salt of hydrochloric acid or a salt of trifluoroacetic acid (TFA):

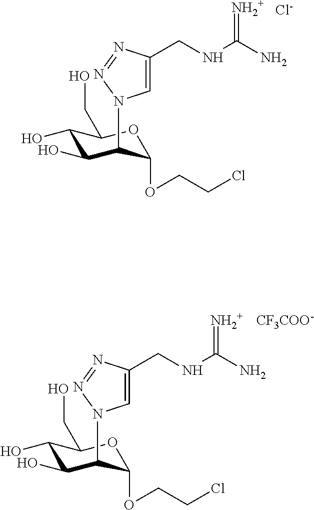

In accordance with an embodiment, the group R¹ has formula (II-A) wherein: n 0, X═NH, m=0, Y═NH. Preferably, Rₐ═H and/or R_{b}═H. Preferably, n=1.

According to a second embodiment, the group R′ has formula (II-B):

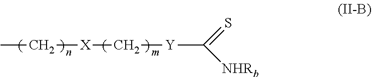

where when n=0, X is CH₂. Preferably, n=0, X═CH₂, m=0, Y═NH, R_{b}═H.

According to a third embodiment, the group R¹ has formula (II-C):

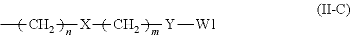

where when n=0, X is CH₂.

In accordance with this embodiment, preferably, X is CH₂ and Y═NH. Preferably, n=0 and/or m=0.

According to different embodiments, the compound (I) according to the present invention is selected from those reported in table 1 below.

TABLE 1

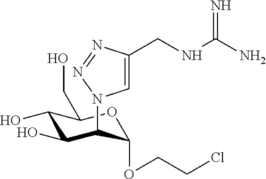

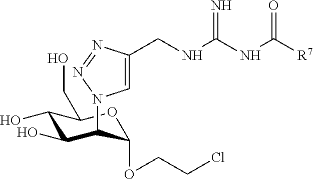

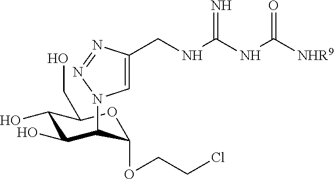

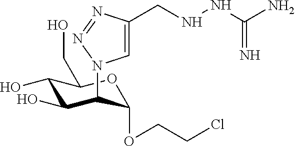

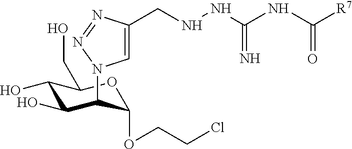

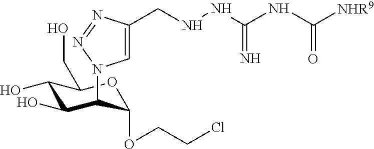

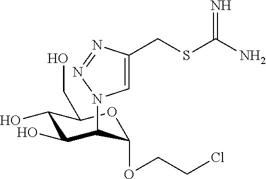

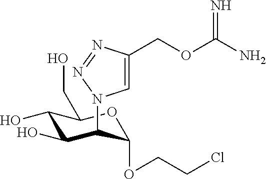

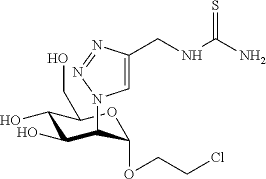

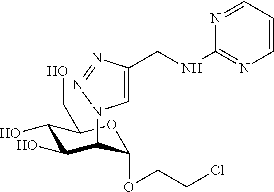

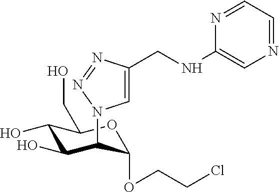

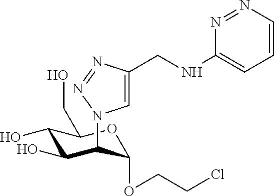

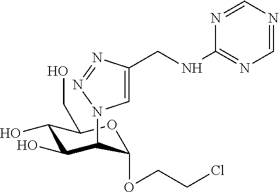

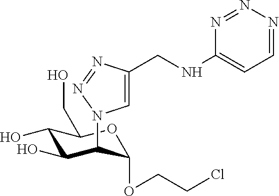

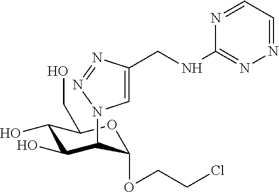

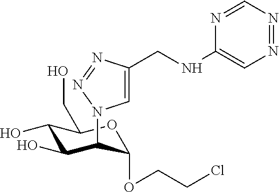

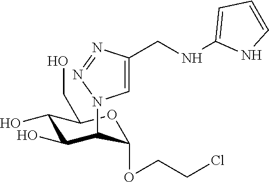

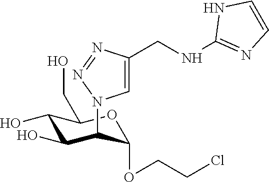

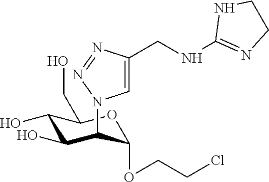

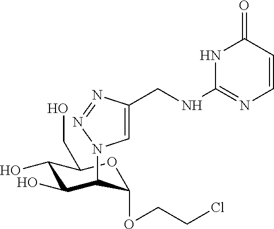

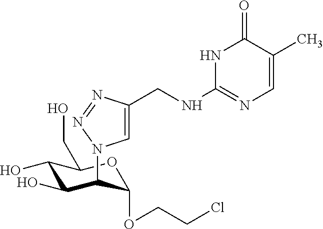

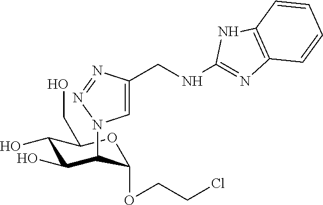

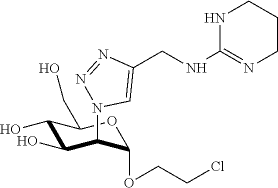

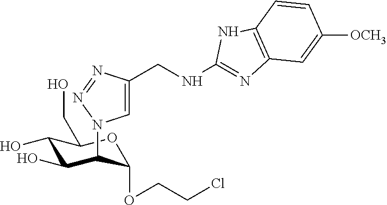

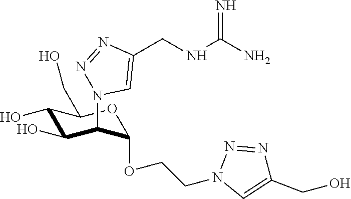

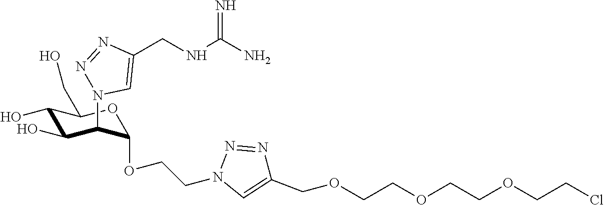

Preferably, the compound (IA) is in the form of a salt, preferably a salt of hydrochloric acid or a salt of trifluoroacetic acid (TFA). Preferably, the compound (IA) has the formula:

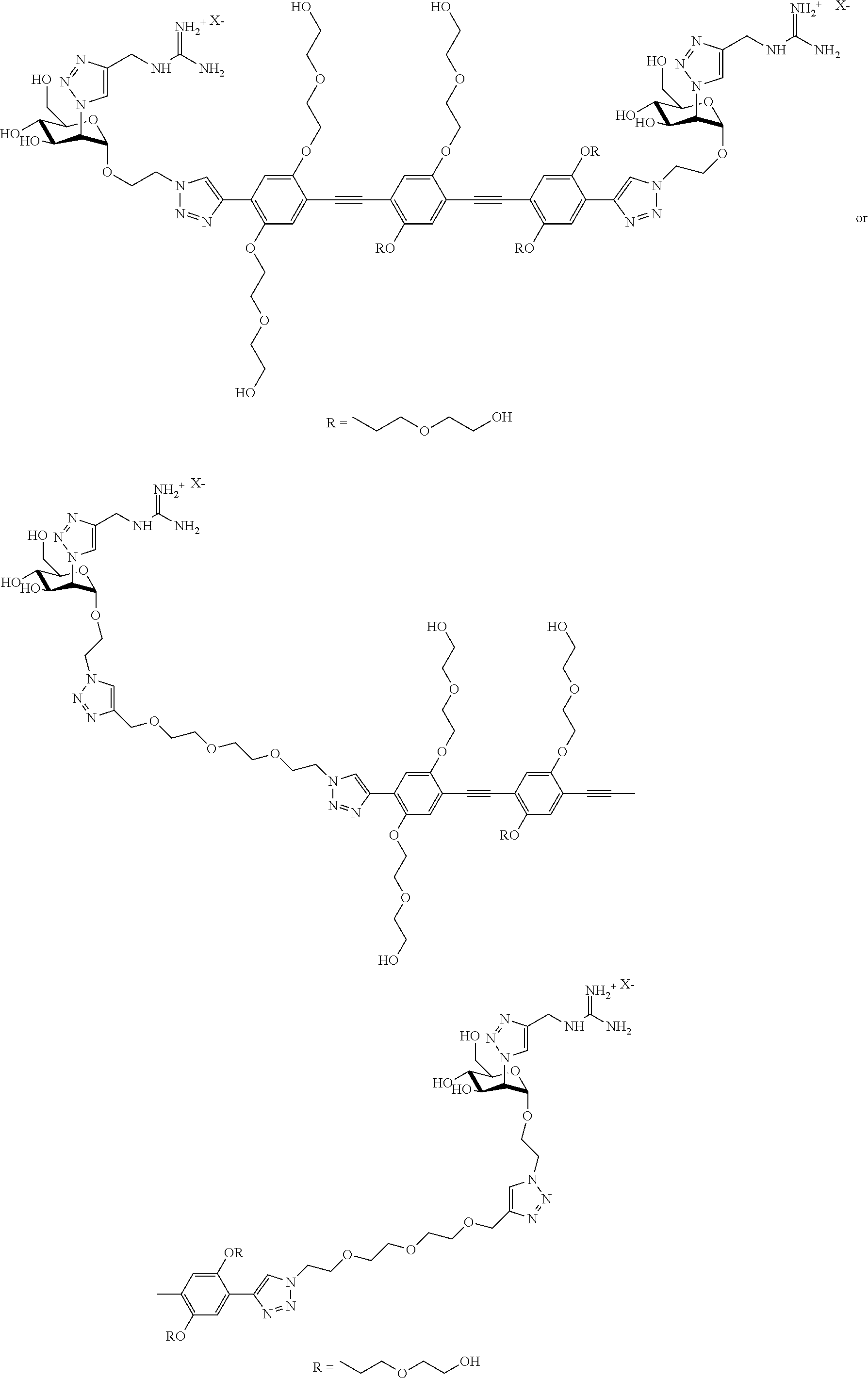

where X⁻═Cl⁻ or CF₃COO⁻.

The present invention also includes compound (I) and compound (IA), as well as their respective salts, for medical use.

In the following description, the expressions “compound (I)” and “compound (IA)” also refer to the salts of the respective compounds.

Compound (I) and compound (IA) act as selective inhibitors of the L-SIGN receptor.

According to one embodiment, compound (I) and compound (IA) are advantageously employed for medical use in the prevention and/or treatment of virus infections which exploit ACE2 as a receptor to enter the target cell.

According to one embodiment, said viruses are the viruses of the Orthocoronavirinae subfamily. For example, said viruses are the SARS-CoV2 virus and its variants. For example, these variants are the WT variants: Wuhan variant, UK: United Kingdom variant (B.1.1.7), SA: South African variant (B.1.53) and BR: Brazilian variant (P.1).

According to further embodiments, said viruses include: West Nile virus (WNV), or hepatitis C virus (HCV), or Zika virus (Zikv), or Ebola virus.

Also, object of the present invention are compound (I) and compound (IA) for medical use in the immunotherapy of liver tumors and/or in the treatment of immune diseases, in which said compounds act as targeting agents of drugs to liver sinusoidal endothelial cells (L-SEC) expressing the L-SIGN receptor. The targeting agents and drugs can be comprised in non-covalent formulations (e.g., liposomes and the like) or the carriers can be covalently linked to the drug using functional elements in the —OR group.

The synthetic methodologies of compounds (I) and (IA) according to the present invention are reported below.

### Method of Synthesis No. 1

A method is described for preparing the compound of formula (I), or a salt thereof, in which R′ is selected from the group consisting of:

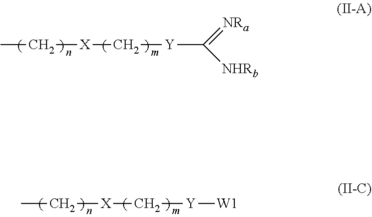

in which:
X═CH₂, Y═NH, Rₐ═H, R_{b}═H, n and m are as defined above.

Said method comprises a nucleophilic substitution reaction between the amino precursor of formula (III):

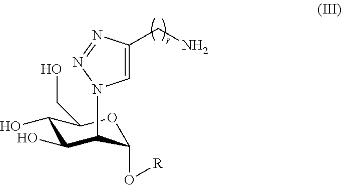

where r is an integer between 1 and 7, preferably 1,
and a molecule having the formula:

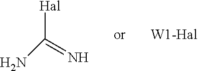

wherein Hal is a halogen, preferably chlorine or bromine, or other leaving group in nucleophilic substitution reactions.

Said amino precursor (1.0 eq) and said molecule comprising a leaving group (1.4 eq) are dissolved in a high boiling solvent, for example DMF, DMSO, NMP, DMA or sulfolane (the concentration of the amino precursor is 0.1-0.5 M), and mixed at a temperature between 8° and 100° C. until completion of the reaction, monitored by TLC. The reaction product is purified by flash chromatography.

According to an embodiment of the invention, the compound (I) in which R¹ has formula (II-A) is in the form of salt and the method for its synthesis comprises a nucleophilic substitution reaction between the amino precursor of formula (III) and formamidine hydrochloride having the formula:

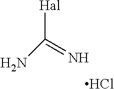

As an alternative to the method described above, the compound (I) in which R¹ has formula (II-A) can be prepared in accordance with the synthetic methodologies illustrated in the experimental part of the present application.

The amino precursor of formula (III) can be obtained starting from an azide 1 according to the following reaction scheme (SCHEME 1):

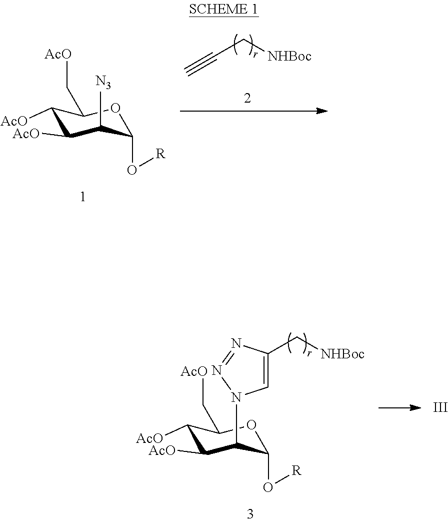

An experimental procedure for obtaining the aforementioned compound is described below in accordance with SCHEME 1.

A 0.04 M CuSO₄·5H₂O solution and a 0.16 M Na-ascorbate solution are prepared in deoxygenated water. A 0.4 M solution of alkyne 2 and a 0.4 M solution of azide 1 are prepared in deoxygenated THF. To the solution of the alkyne 2 (1 mol eq) are added, in sequence: 0.1 mol eq of the CuSO₄·5H₂O solution; 0.4 mol eq of the Na-ascorbate solution; 1 mol eq of the azide 1 solution. The reaction mixture is stirred at room temperature, under N₂ and protected from light. At the end of the reaction, monitored by TLC, the solvents are evaporated and the crude triazole 3 is purified by automated flash chromatography to obtain a white foam.

Triazole 3 is dissolved in anhydrous methanol, then a freshly prepared 0.1 M NaOMe solution is added. The reaction mixture is stirred at room temperature for one hour. Upon completion (monitoring by TLC), the reaction is neutralized 5 with Amberlite® IR120 hydrogen form ion-exchange resin, filtered and concentrated under vacuum. The crude product is dissolved in a 4:1 mixture of anhydrous CH₂—Cl₂ and TFA. The reaction is stirred at room temperature, under N₂ for one hour. At the end of the reaction (monitoring by TLC), the mixture is concentrated under vacuum and co-evaporated with toluene three times to obtain the amino precursor (III) as the TFA salt.

### Method of Synthesis No. 2

A method is described for preparing the compound of formula (I), or a salt thereof, in which R′ is:

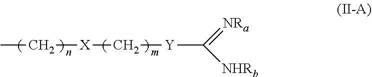

in which:
X═CH₂; Y═S or O; Rₐ═H; R_{b}═H, n and m are as defined above.

As shown in the reaction scheme below (SCHEME 2), said method comprises a cycloaddition reaction between azide 1 and a terminal alkyne 4 to give the corresponding triazole 5; an addition reaction between triazole 5 and cyanamide to give compound 6; deacetylation of compound 6 to give product 7.

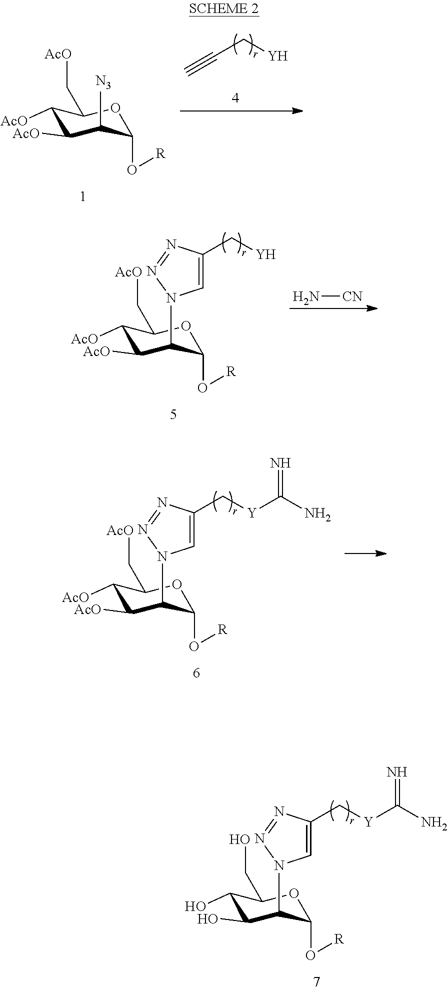

in which:
r is an integer between 1 and 7, preferably 1,
Y is S or O.

An experimental procedure for obtaining the aforementioned compound in accordance with SCHEME 2 is described below.

A 0.04 M CuSO₄·5H₂O solution and a 0.16 M Na-ascorbate solution are prepared in deoxygenated water. A 0.4 M solution of the terminal alkyne 4 and a 0.4 M solution of azide 1 in are prepared deoxygenated THF. To the solution of the terminal alkyne 4 (1.0 mol eq) are added, in sequence: 0.1 mol eq of the CuSO₄·5H₂O solution; 0.4 mol eq of the Na-ascorbate solution; 1 mol eq of the azide 1 solution. The reaction mixture is stirred at room temperature, under N2 and protected from light. At the end of the reaction, monitored by TLC, the solvents are evaporated and the crude product is purified by flash chromatography to obtain pure triazole 5.

When Y═O: a mixture of triazole 5 (2.0 eq), cyanamide (1.2 eq) and anhydrous p-toluenesulfonic acid (1.3 eq) in anhydrous chloroform (1 M) is stirred under nitrogen until completion of the reaction; the mixture is then neutralized with triethylamine and the solvent removed at reduced pressure, the crude product is purified by flash chromatography to obtain compound 6.

When Y═S: a catalytic amount of triethylamine (0.1 eq) is added to a mixture of triazole 5 (1.0 eq) and cyanamide (1.2 eq) in diethyl ether (1 M). After 2 hours of reflux, the reaction mixture is cooled to room temperature and stirred to completion (TLC monitoring); the solvent is then removed under reduced pressure and the crude product purified by flash chromatography to obtain compound 6.

To a solution of product 6 (1.0 eq) in dry methanol (where Y is O or S) a freshly prepared 0.1 M NaOMe solution in dry methanol is added to a 0.05 M final concentration of the substrate (0.014 M final concentration of NaOMe). The reaction mixture is stirred at room temperature, under N₂ for one hour. Upon completion (TLC monitoring), the reaction is neutralized with Amberlite® IR120 hydrogen form ion-exchange resin, filtered and concentrated in vacuo to obtain the pure product 7.

### Method of Synthesis No. 3

A method is described for preparing the compound of formula (I), or a salt thereof, in which R¹ is:

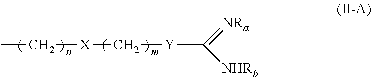

in which:
X═CH₂ or NH, m=0, Y═NH, with the condition that when n=0, X is CH₂; Rₐ═H, R_{b}═H; n and m are as defined above.

Said method comprises an addition reaction between 1,3-Di-Boc-2-(trifluoromethylsulfonyl)guanidine and compound 8 to give compound 9; a cycloaddition reaction between azide 1 and compound 9 to give triazole 10; deacetylation of compound 10 to give compound 11; removal of the Boc protecting group with an acid to give product 12, as shown in the following reaction scheme (SCHEME 3):

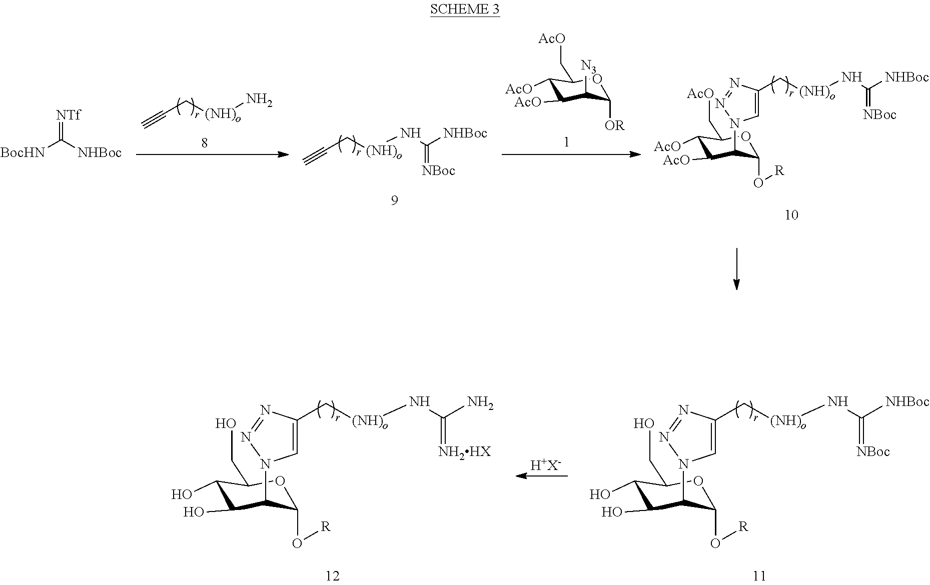

where r is an integer between 1 and 7, preferably 1,
o is an integer selected between 0 and 1,
preferably, X⁻ is Cl⁻ or CF₃COO⁻.

An experimental procedure for obtaining the above compound is described below in accordance with SCHEME 3.

1,3-Di-Boc-2-(trifluoromethylsulfonyl)guanidine (0.9 eq) is dissolved in anhydrous dichloromethane (0.2 M), then triethylamine (1 eq) and compound 8 (1 eq) are added; examples of compound 8 are propargylamine and propargylhydrazine. The reaction mixture is stirred at room temperature, under nitrogen. Once the reaction is completed (TLC monitoring), the mixture is concentrated under vacuum and purified by flash chromatography to obtain compound 9.

A 0.04 M CuSO₄·5H₂O solution and a 0.16 M Na-ascorbate solution are prepared in deoxygenated water. A 0.6 M solution of compound 9 and a 0.4 M solution of azide 1 are prepared in deoxygenated THF. To the solution of compound 9 (1.5 mol eq) are added, in sequence: 0.1 mol eq of the CuSO₄·5H₂O solution; 0.4 mol eq of the Na-ascorbate solution; 1 mol eq of the azide 1 solution. At the end of the reaction, monitored by TLC, the solvents are evaporated and the crude product is purified by flash chromatography to obtain pure triazole 10.

To a solution of triazole 10 (1.0 eq) in anhydrous methanol, a freshly prepared 0.1 M NaOMe solution in anhydrous MeOH is added until a final concentration of 0.05 M of the substrate is reached (final concentration of NaOMe of 0.014 M). The reaction mixture is stirred at room temperature, under N₂ for one hour. Upon completion (TLC monitoring), the reaction is neutralized with Amberlite® IR120 hydrogen form ion-exchange resin, filtered and concentrated under vacuum to obtain the pure product 11.

When X⁻═CF₃COO⁻: compound 11 (1 eq) is dissolved in a 4:1 mixture of anhydrous CH₂Cl₂ and TFA (0.05 M). The reaction mixture is stirred at room temperature, under N₂ for 5 hours. Upon completion (TLC monitoring), the mixture is concentrated under vacuum and co-evaporated with toluene three times to obtain pure product 12.

When X⁻═Cl⁻: compound 11 (1 eq.) is dissolved in CH₂Cl₂ (0.04 M); then 3 mol eq are added of a 1 M aqueous solution of HCl. The reaction mixture is stirred at room temperature for 3 days. Upon completion (TLC monitoring), the mixture is concentrated under vacuum and co-evaporated with toluene three times to obtain pure product 12.

### Method of Synthesis No. 4

A method is described for preparing the compound of formula (I), or a salt thereof, in which R¹ is:

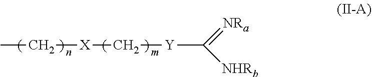

in which:
X═CH₂ or NH, m=0, Y═NH, with the condition that when n=0, X is CH₂,
Rₐ and/or R_{b} are independently selected from COR⁷, COOR⁸, CONHR⁹, where R⁷, R⁸ and R⁹ are as defined above.

Said method provides for the synthesis of compound 11 as described with reference to the previous SCHEME 3; compound 11 is reacted with a nucleophilic agent to give compound 13, which is subsequently subjected to removal of the protective group Boc with an acid to give product 14a/14b (tautomers), as shown in the following reaction scheme (SCHEME 4):

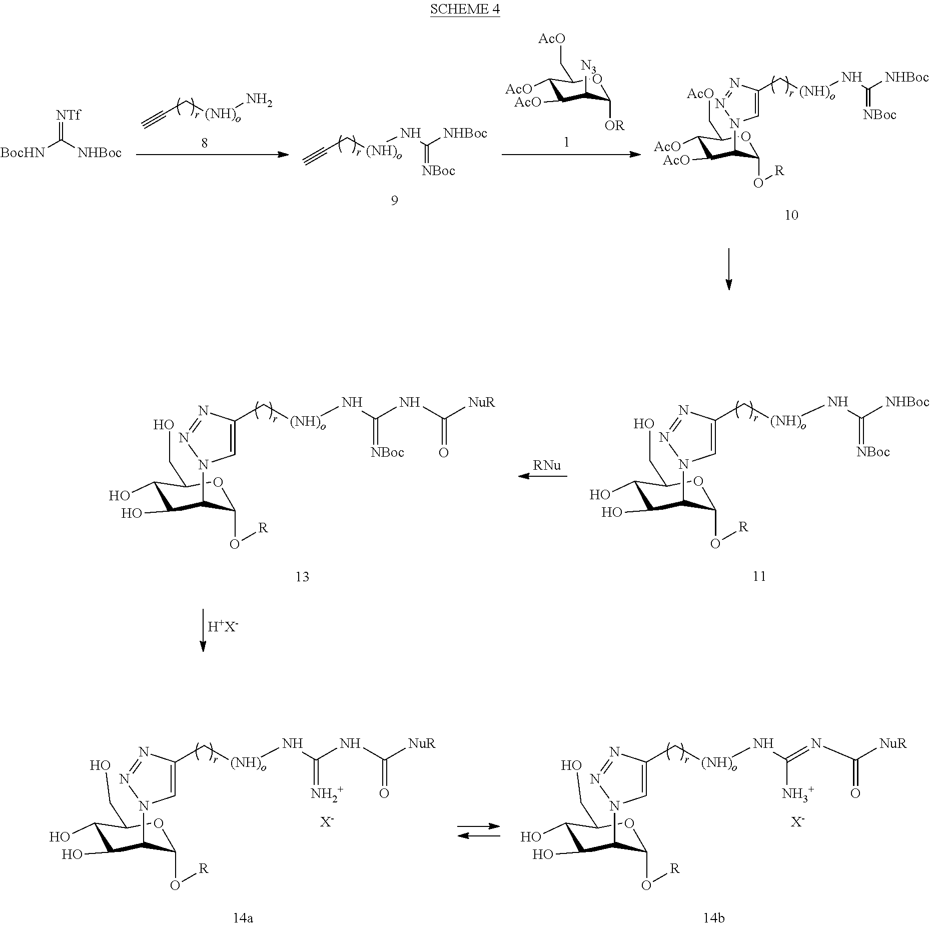

in which:
r is an integer between 1 and 7, preferably 1,
o is an integer selected between 0 and 1,
RNu is a nucleophilic agent, in which:
R is H, a C₁-C₆ alkyl, C₃-C₆ cycloalkyl or a C₆-C₁₀ aryl, and Nu is for example S⁻, O⁻, —NH₂,
preferably, X⁻ is Cl⁻ or CF₃COO⁻.

As a nucleophilic agent, a Grignard reagent can also be employed.

An experimental procedure for obtaining the aforesaid compound in accordance with SCHEME 4 is described below. Compound 11 is obtained in accordance with the description provided regarding SCHEME 3, which will not be repeated here.

A solution of compound 11 (1 eq) and nucleophilic agent (2 eq) in refluxed THF (0.3 M) is stirred until reaction is complete (TLC monitoring). The resulting solution is then concentrated under reduced pressure and the crude product purified by flash chromatography to obtain compound 13.

When X⁻ ═CF₃COO⁻: compound 13 (1 eq.) is dissolved in a 4:1 mixture of anhydrous CH₂Cl₂ and TFA (0.05 M). The reaction mixture is stirred at room temperature, under N₂ for 5 hours. Upon completion (TLC monitoring), the mixture is concentrated under vacuum and co-evaporated with toluene three times to obtain the product 14a/14b (tautomers).

When X⁻═Cl⁻: compound 13 (1 eq.) is dissolved in CH₂Cl₂ (0.04 M); then 3 mol eq are added of a 1 M aqueous solution of HCl. The reaction mixture is stirred at room temperature for 3 days. At the end of the reaction (TLC monitoring), the mixture is concentrated under vacuum and co-evaporated with toluene three times to obtain the product 14a/14b (tautomers).

A specific example of the above method is reported below, 5 in which compound 11 reacts with the nucleophilic agent RNH₂ to give compound 13′, which in turn is treated in an acidic environment to give the amidinourea 14a′/14b′ (tautomers), according to the following reaction (SCHEME 4A). The reaction conditions are as reported above.

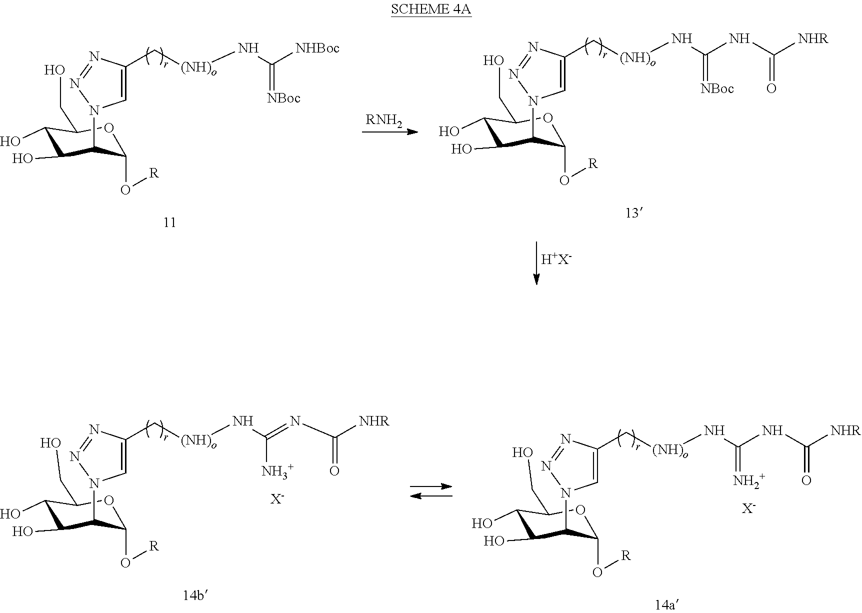

### Method of Synthesis No. 5

A method is described for preparing the compound of formula (I), or a salt thereof, in which R¹ is:

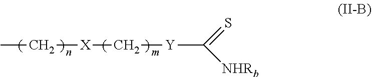

where X═CH₂, Y═NH, R_{b}═H.

Said method comprises an addition reaction between alkyne and ammonium thiocyanate to give compound 16; a cycloaddition reaction between compound 16 and azide 1 to give the corresponding triazole 17; a deacetylation reaction to give product 18, as shown in the following reaction scheme (SCHEME 5).

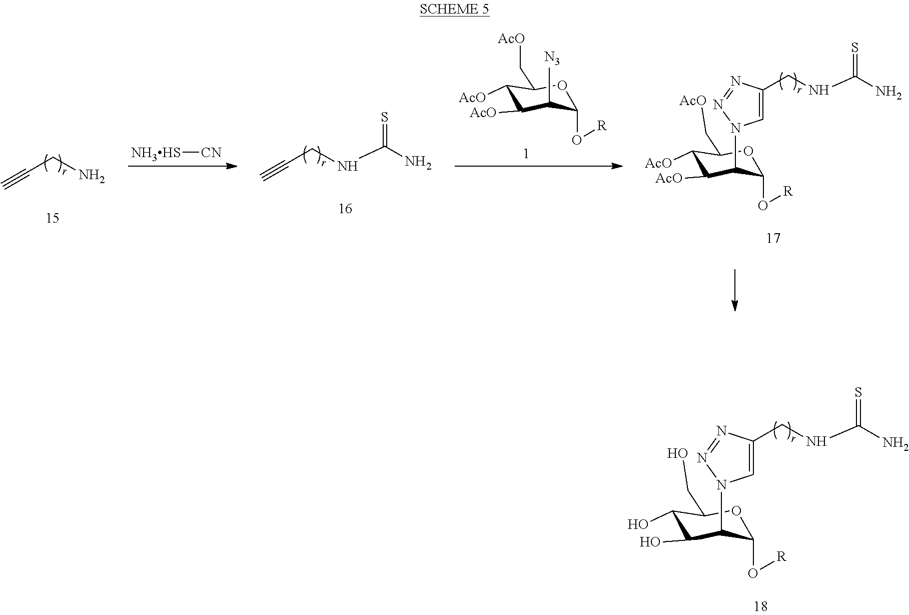

where r is an integer between 1 and 7, preferably 1.

An experimental procedure for obtaining the aforementioned compound in accordance with SCHEME 5 is described below.

Compound 15 (1.0 eq) is dissolved in water (2 M) and 3 eq of concentrated HCl; an example of compound 15 is propargylamine. The resulting solution is heated until dissolved, then cooled to room temperature. Ammonium thiocyanate (1.0 eq) is added to the solution and heated to 90° C. overnight. At the end of the reaction (monitoring by TLC), the solution is cooled to room temperature and concentrated under reduced pressure. The crude product is purified by flash chromatography to obtain compound 16.

A 0.04 M CuSO₄·5H₂O solution and a 0.16 M Na-ascorbate solution are prepared in deoxygenated water. A 0.6 M solution of compound 16 and a 0.4 M solution of azide 1 are prepared in deoxygenated THF. To the solution of compound 16 (1.5 mol eq) are added, in sequence: 0.1 mol eq of the CuSO₄·5H₂O solution; 0.4 mol eq of the Na-ascorbate solution; 1 mol eq of the azide 1 solution. The reaction mixture is stirred at room temperature, under N₂ and protected from light. At the end of the reaction, monitored by TLC, the solvents are evaporated and the crude product is purified by flash chromatography to obtain pure triazole 17.

A freshly prepared 0.1 M NaOMe solution in anhydrous methanol is added to a solution of triazole 17 (1.0 eq) in anhydrous methanol until a final substrate concentration of 0.05 M is reached (final NaOMe concentration of 0.014 M). The reaction mixture is stirred at room temperature, under N₂. Upon completion (monitoring by TLC), the reaction is neutralized with Amberlite® IR120 hydrogen form ion-exchange resin, filtered and concentrated under vacuum to obtain pure product 18.

### Method of Synthesis No. 6

A method is described for preparing the compound of formula (I), or a salt thereof, in which R is selected from the group consisting of C₁-C₆ alkyls, C₃-C₆ cycloalkyls, C₆-C₁₀ aryls, R²Cl, where R² is as defined above.

Said method provides for the introduction of the aforementioned R group through the glycosylation reaction of an R—OH alcohol, preferably primary, with the 2-azido mannosyl donor 19a, as reported in the following reaction scheme (SCHEME 6A).

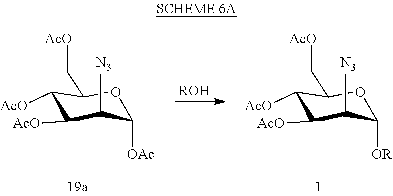

An experimental procedure for obtaining azide 1 in accordance with SCHEME 6A, in which ROH is 2-chloroethanol (R=—CH₂CH₂Cl), is described below.

Compound 19a (1.0 eq) is dissolved in anhydrous CH₂Cl₂ (concentration of compound 19a=0.5 M), then 2-chloroethanol (2.0 eq) is added. The reaction mixture is cooled to 0° C., then BF₃·Et₂O (4.0 eq) is added. The reaction is stirred at room temperature, under N₂ overnight. At the end of the reaction (monitoring by TLC), the mixture is extracted twice with cold water, once with a saturated solution of NaHCO₃ and once again with cold water. The organic phase is dried over Na₂SO₄, filtered and concentrated under vacuum. The crude product is purified by automated flash chromatography.

Alternatively, the aforementioned R—OH alcohol glycosylation reaction can use the donor 2-azido mannosyl 19b as reagent, as reported in the following reaction scheme (SCHEME 6B).

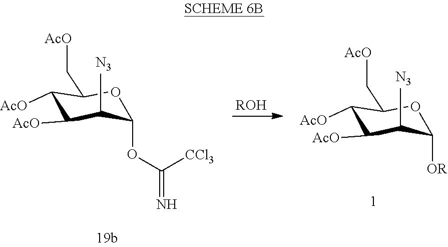

An experimental procedure for obtaining azide 1 according to SCHEME 6B, in which ROH is 2-chloroethanol (R═—CH₂CH₂Cl), is described below.

Compound 19b (1.0 eq) and activated and powdered molecular sieves 4A are co-evaporated with toluene three times and left under vacuum overnight. The mixture is then dissolved in anhydrous CH₂Cl₂ (concentration of compound 19b=0.1 M), 2-chloroethanol (1.0 eq) is added and the solution is cooled to −30° C.; then TMSOTf (0.2 eq) is added. The mixture is stirred at −30° C. until completion (TLC monitoring). Upon completion, the mixture is quenched with TEA (0.4 eq) and concentrated under vacuum. The crude product is purified by flash chromatography.

The azide 1 resulting from the aforementioned alcohol glycosylation reactions is subjected to the methods 1-5 described above, to obtain the compound according to the invention.

### Method of Synthesis No. 7

A method is described for preparing the compound of formula (I), or a salt thereof, wherein R is —R³NH₂, wherein R³ is as defined above.

Said method provides for the formation of azide 21 from the respective chloride 20 and the reduction of azide 21 to amine 22, in accordance with SCHEME 7, which gives the specific example in which R³ is a C₁-C₆ alkylene (with z integer between 1 and 6). The chloride 20 can be obtained with the method of SCHEME 6A or 6B.

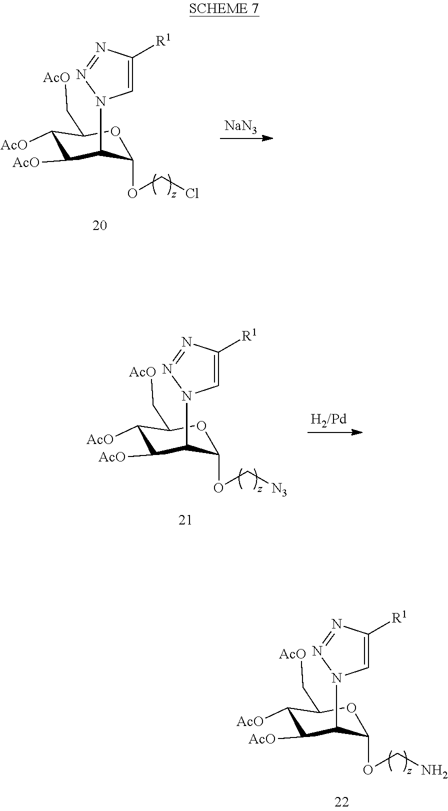

An experimental procedure for obtaining the amine 22 in accordance with SCHEME 7 is described below.

Compound 20 (1.0 eq) is dissolved in anhydrous DMF, then NaN₃ (5.0 eq) is added. The reaction mixture is stirred at 55° C. for 3 days. At the end of the reaction (monitoring by ¹H-NMR), the mixture is concentrated under vacuum and the crude compound 21 purified by automated flash chromatography to obtain pure compound 21.

Compound 21 (1.0 eq) is dissolved in anhydrous methanol (concentration of compound 21=0.05 M) and 10% Pd/C or Pd Lindlar is added. The reaction is stirred in an atmosphere of H₂ (1 atm) at room temperature. At the end of the reaction (monitoring by TLC), the catalyst is filtered on celite and the solvent is evaporated under reduced pressure to obtain pure product 22.

The product 22 is then subjected to deacetylation with a NaOMe solution, in accordance with the procedure described above in relation to SCHEMES 2-5.

In the synthesis method according to SCHEME 7, as a starting substrate, as an alternative to the acetylated compound 20, it is possible to use the corresponding deacetylated molecule.

As an alternative to the method according to SCHEME 7, it is possible to introduce the aforementioned R group starting from a reaction intermediate, for example in accordance with the following reaction scheme (SCHEME 7A):

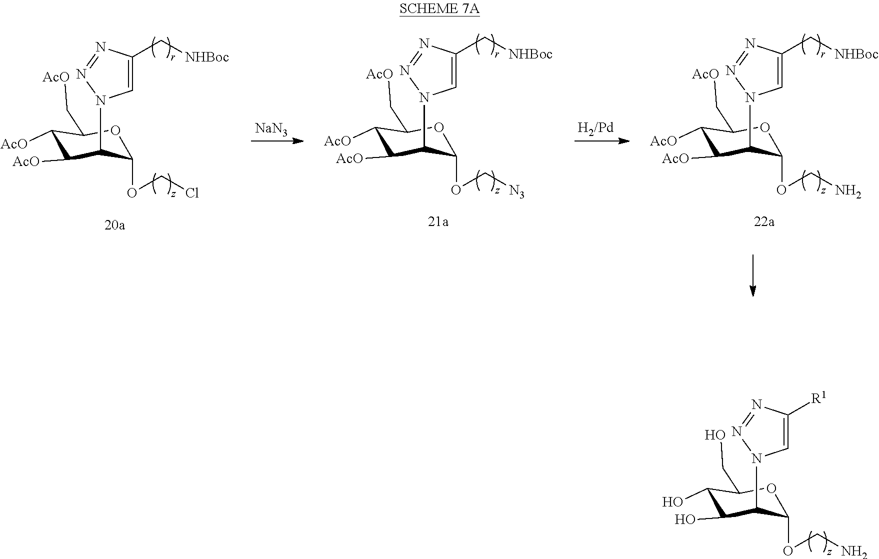

In the example illustrated in the above scheme, the intermediate 20a reacts with NaN₃ to give the corresponding azide 21a, which is reduced to amine 22a. Subsequently, the compound 22a reacts according to the SCHEME 1 described above to give the amino precursor of formula (III) (not shown in the above scheme), which is then subjected to a nucleophilic substitution reaction according to the synthesis method no. 1 to give the reaction product.

### Method of Synthesis No. 8

A method for preparing the compound of formula (I), or a salt thereof, is described, in which R is —R⁴NHC(O) R′, in which R⁴ and R′ are as defined above.

Said method provides for the reaction of the amine 22 obtained with the method of SCHEME 7 with an acyl chloride R′COCl or a carboxylic acid R′COOH, in accordance with SCHEME 8.

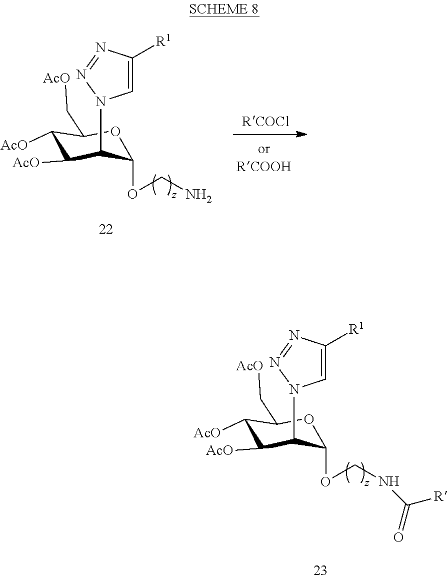

An experimental procedure for obtaining amide 23 in accordance with SCHEME 8 is described below.

Reaction with R′COCl acyl chloride: sodium hydride (2.0 eq) is added at 0° C. to a solution of compound 22 (1.0 eq) in 1,4-dioxane (concentration of compound 22=1.5 M), kept under stirring. Subsequently the acyl chloride (1.0 eq) is added dropwise. The resulting mixture is stirred at room temperature. At the end of the reaction (monitoring by TLC) the mixture is extracted with CH₂Cl₂ and the organic phase is washed with water, then dried over Na₂SO₄, filtered and concentrated under vacuum. The crude product 23 is purified by flash chromatography to obtain the pure product 23.

Reaction with carboxylic acid R′COOH: compound 22 (1.0 eq) is dissolved in anhydrous THF (0.2 M). Carboxylic acid (1.2 eq) and DCC (1.2 eq) are added in this order. The resulting mixture is stirred at room temperature under N₂. At the end of the reaction (monitoring by TLC), the mixture is filtered on celite and washed with CH₂Cl₂. The crude product 23 is purified by flash chromatography to obtain the pure product 23.

The product 23 is then subjected to deacetylation with a NaOMe solution, in accordance with the procedure described above in relation to SCHEMES 2-5.

In the synthesis method according to SCHEME 8, as a starting substrate, as an alternative to the acetylated compound 22, it is possible to use the corresponding deacetylated molecule.

As an alternative to the method according to SCHEME 8, it is possible to introduce the aforementioned R group starting from a reaction intermediate, for example in accordance with the following reaction scheme (SCHEME 8A):

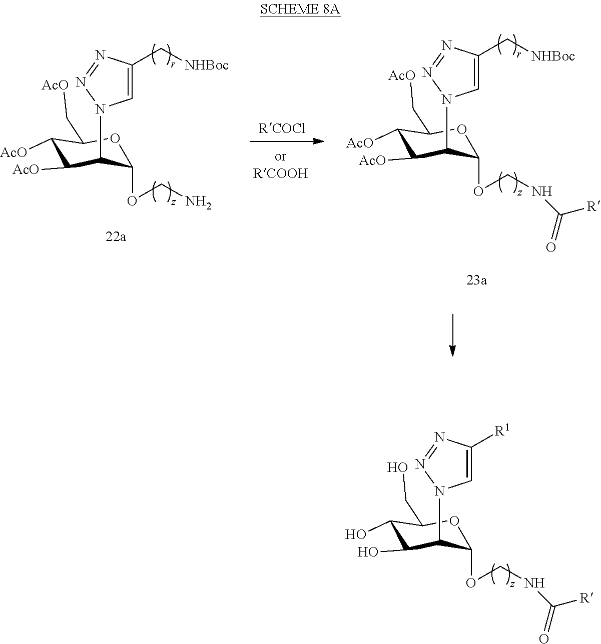

In the example illustrated in the above scheme, the intermediate 22a reacts with an acyl chloride or a carboxylic acid to give the amide 23a. Subsequently, the compound 23a reacts according to the SCHEME 1 described above to give the amino precursor of formula (III) (not shown in the above scheme), which is then subjected to a nucleophilic substitution reaction according to the synthesis method no. 1 to give the reaction product.

### Method of Synthesis No. 9

A method is described for preparing the compound of formula (I), or a salt thereof, in which R is —R³NH₂, —R⁵SR″ or —R⁶OR″, in which R³, R⁵, R⁶ e R″ are as defined above.

Said method provides for a nucleophilic substitution reaction between compound 20 and a nucleophilic agent, in accordance with SCHEME 9, which gives the specific example in which R³, R⁵, R⁶ is a C₁-C₆ alkylene (with z being an integer between 1 and 6):

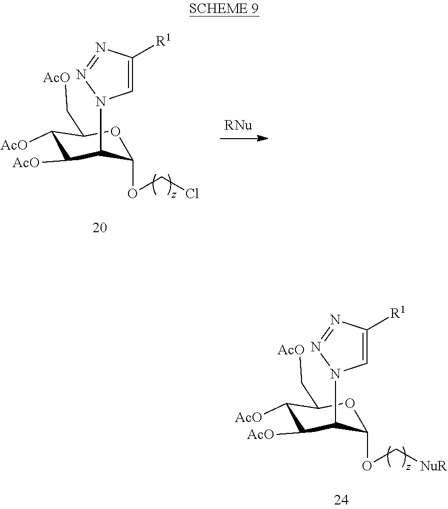

in which:
RNu is a nucleophilic agent, in which:
R is H, a C₁-C₆ alkyl, a C₃-C₆ cycloalkyl or a C₆-C₁₀ aryl, and Nu is selected from S⁻, O⁻ and —NH₂.

An experimental procedure for obtaining the product 24 in accordance with SCHEME 9, in which the nucleophilic agent is a thiolate, is described below.

Compound 20 (1.0 eq) is dissolved in anhydrous DMF (0.2 M), then the corresponding thiol R″ SH (5 eq) is added. The resulting reaction mixture is stirred at 55° C. for 3 days. At the end of the reaction (monitoring by ¹H-NMR), the mixture is concentrated under vacuum and the crude product 24 is purified by automated flash chromatography to obtain pure product 24.

The product 24 is then subjected to deacetylation with a NaOMe solution, in accordance with the procedure described above in relation to SCHEMES 2-5.

In the synthesis method according to SCHEME 9, as a starting substrate, as an alternative to the acetylated compound 20, it is possible to use the corresponding deacetylated molecule.

As an alternative to the method according to SCHEME 9, it is possible to introduce the aforementioned R group starting from a reaction intermediate, for example in accordance with the following reaction scheme (SCHEME 9A), which reports the specific example in which the agent nucleophile is a thiolate:

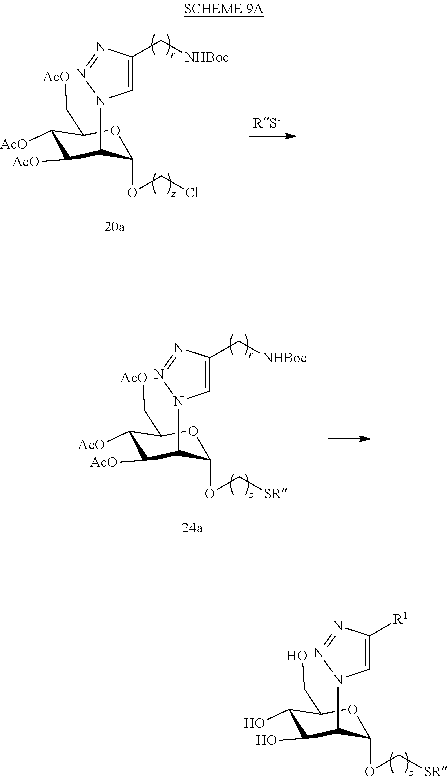

In the example illustrated in the above scheme, the intermediate 20a reacts with the nucleophilic agent to give the compound 24a, which reacts according to the SCHEME 1 described above to give the amino precursor of formula (III) (not shown in the above scheme), which is then subjected to a nucleophilic substitution reaction according to the synthesis method no. 1 to give the reaction product.

### Method of Synthesis No. 10

A method is described for preparing the compound of formula (I), or a salt thereof, in which R is

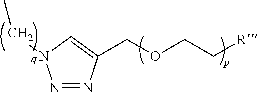

where p, q and R″′ are as defined above.

Said method comprises the formation of an azide 26 starting from the corresponding chloride 25 in the presence of sodium azide; deacetylation of azide 26 to give compound 27; reaction of compound 27 with a terminal alkyne 28 to give product 29, as shown in the following reaction scheme (SCHEME 10)

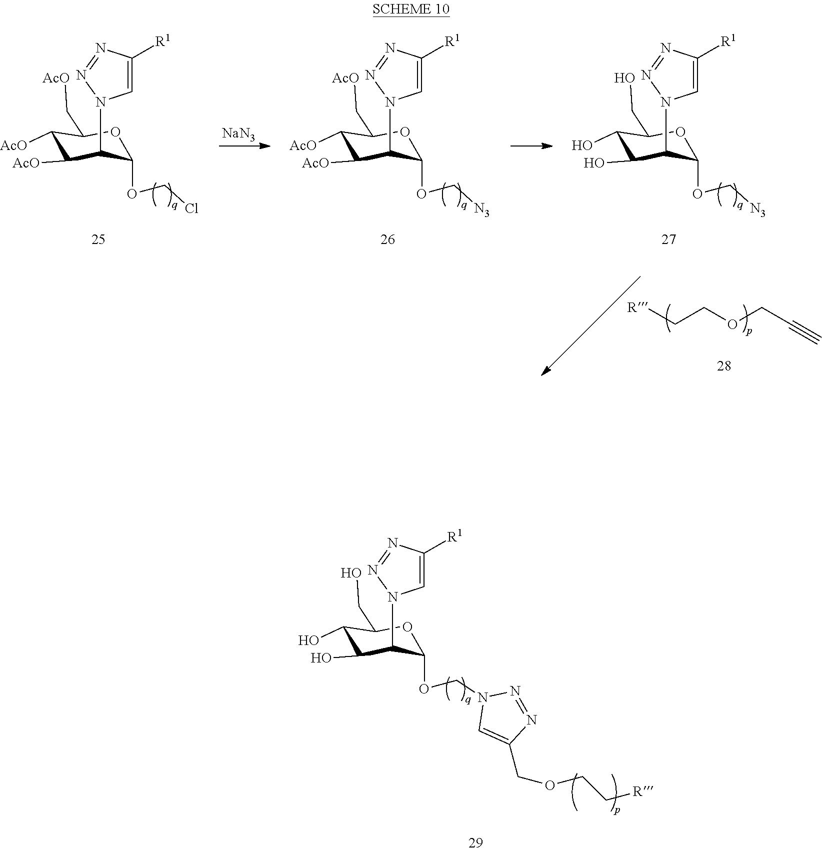

An experimental procedure for obtaining the product 29 in accordance with SCHEME 10 is described below.

Compound 25 (1 eq) is dissolved in anhydrous DMF, then NaN₃ (5 eq) is added. The reaction mixture is stirred at 55° C. for 3 days. At the end of the reaction (monitoring by ¹H-NMR), the mixture is concentrated under vacuum and the crude product is purified by automated flash chromatography to obtain pure compound 26 as a white foam.

A freshly prepared 0.1 M NaOMe solution in anhydrous methanol is added to a solution of 26 (0.07 mmol) in anhydrous methanol until a final substrate concentration of 0.05 M is reached (final NaOMe concentration of 0.014 M). The reaction mixture is stirred at room temperature, under N₂ for one hour. Upon completion (monitoring by TLC), the reaction is neutralized with Amberlite® IR120 hydrogen form ion-exchange resin, filtered and concentrated under vacuum to obtain the pure product 27 as a white foam.

A 0.01 M CuSO₄·5H₂O solution and a 0.04 M Na-ascorbate solution are prepared in deoxygenated water. A 0.04 M solution of alkyne 28 (according to Adibekian et al Nat. Chem. Biol. 2011, 7, 469), a 0.03 M solution of Tris[(1-benzyl-1H-1,2,3-triazol-4-yl)methyl]amine (TBTA) and a 0.2 M solution of compound 27 are prepared in deoxygenated THF.

To the alkyne 28 solution (1 mol eq) are added, in sequence: 0.2 mol eq of the TBTA solution; 0.1 mol eq of the CuSO₄·5H₂O solution; 0.4 mol eq of the Na-ascorbate solution.

The mixture is stirred at room temperature for 10 minutes, then 1.0 mol eq of the compound 27 solution is added. The reaction mixture is stirred at room temperature in the dark overnight. At the end of the reaction (monitoring by TLC), the solvents are evaporated and the crude product purified by reverse-phase automated flash chromatography to obtain pure product 29 as a foam.

As an alternative to the method according to SCHEME 10, it is possible to introduce the aforementioned R group starting from a reaction intermediate, for example in accordance with the following reaction scheme (SCHEME 10A):

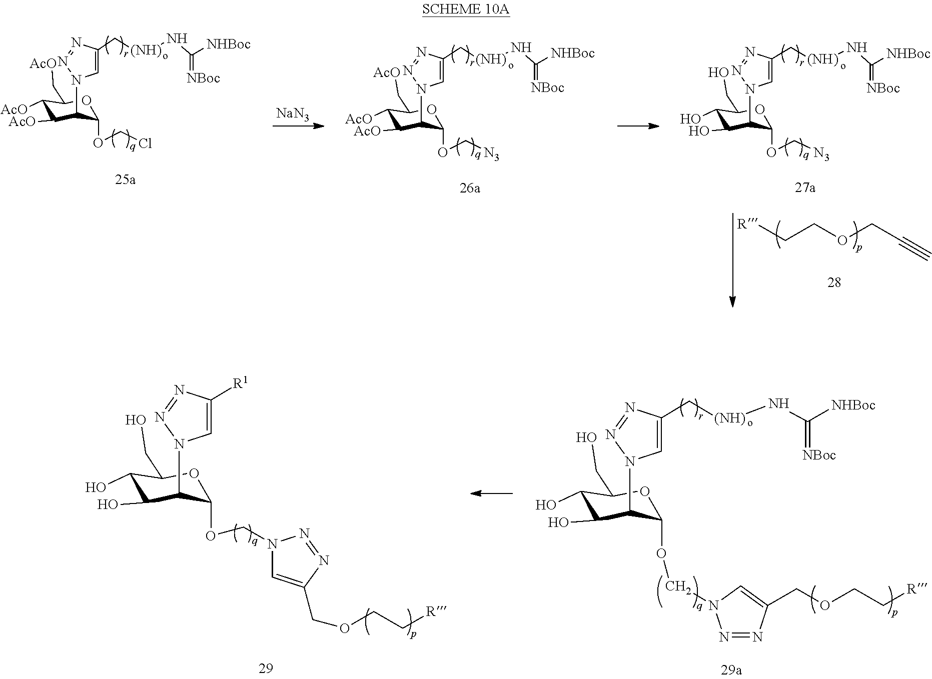

In the example illustrated in the above scheme, the intermediate 25a reacts with NaN₃ to give the corresponding azide 26a, which is deacetylated to give the compound 27a, which in turn reacts with the alkyne 28 to give the compound 29a. The latter is subjected to removal of the Boc protecting group with an acid to give the product 29, or it first reacts with a nucleophilic agent and then is subjected to removal of the Boc protecting group in accordance with SCHEME 4 to give the product 29.

A further method for introducing the aforementioned R group uses the intermediate 25b as a starting substrate, in accordance with the following reaction scheme (SCHEME 10B):

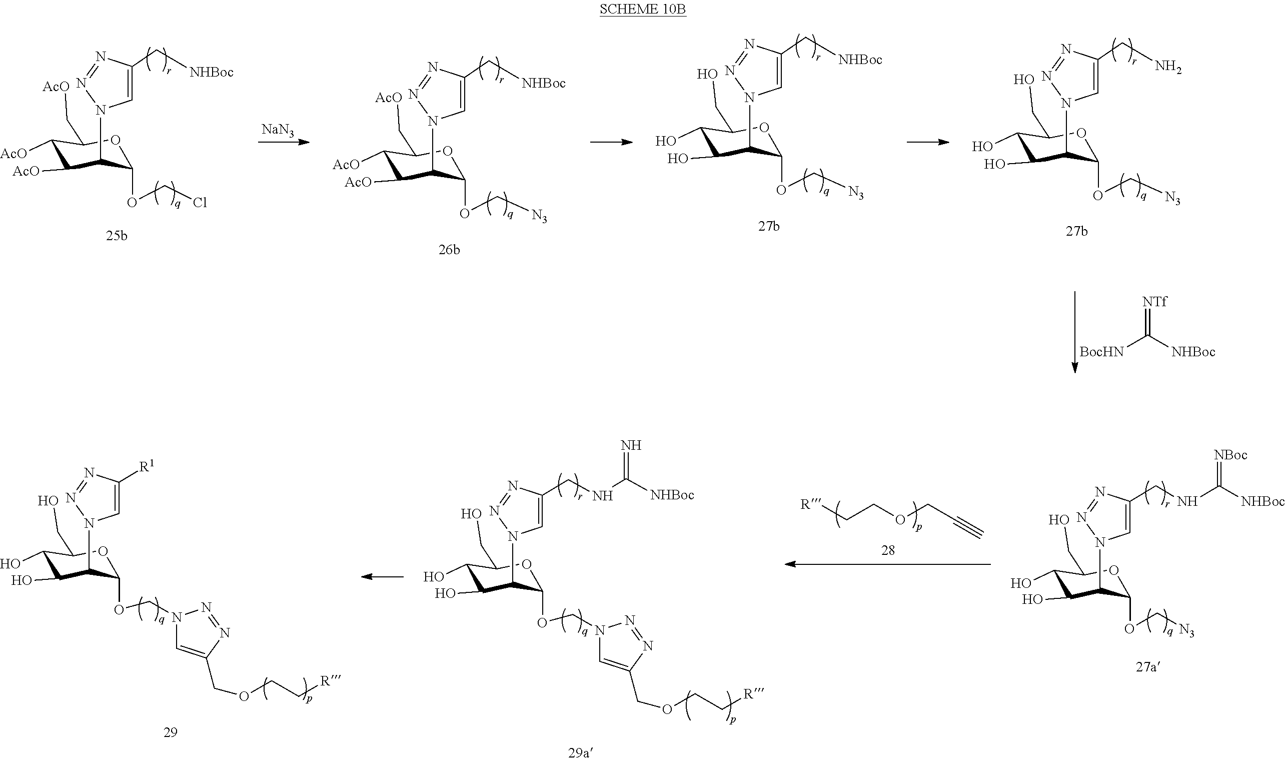

In the example illustrated in the above scheme, the intermediate 25b reacts with NaN₃ to give the corresponding azide 26b, which is deacetylated to give the compound 27b. The latter is subjected to removal of the Boc protecting group with an acid to give compound 27b′, which in turn reacts with 1,3-Di-Boc-2-(trifluoromethylsulfonyl)-guanidine to give compound 27a′. The latter reacts with the alkyne 28 to give the compound 29a′. The latter is subjected to removal of the Boc protecting group with an acid to give the product 29, or it first reacts with a nucleophilic agent and then is subjected to removal of the Boc protecting group in accordance with SCHEME 4 to give the product 29.

A further method for introducing the aforementioned R group uses the intermediate 26b as a starting substrate, in accordance with the following reaction scheme (SCHEME 10C):

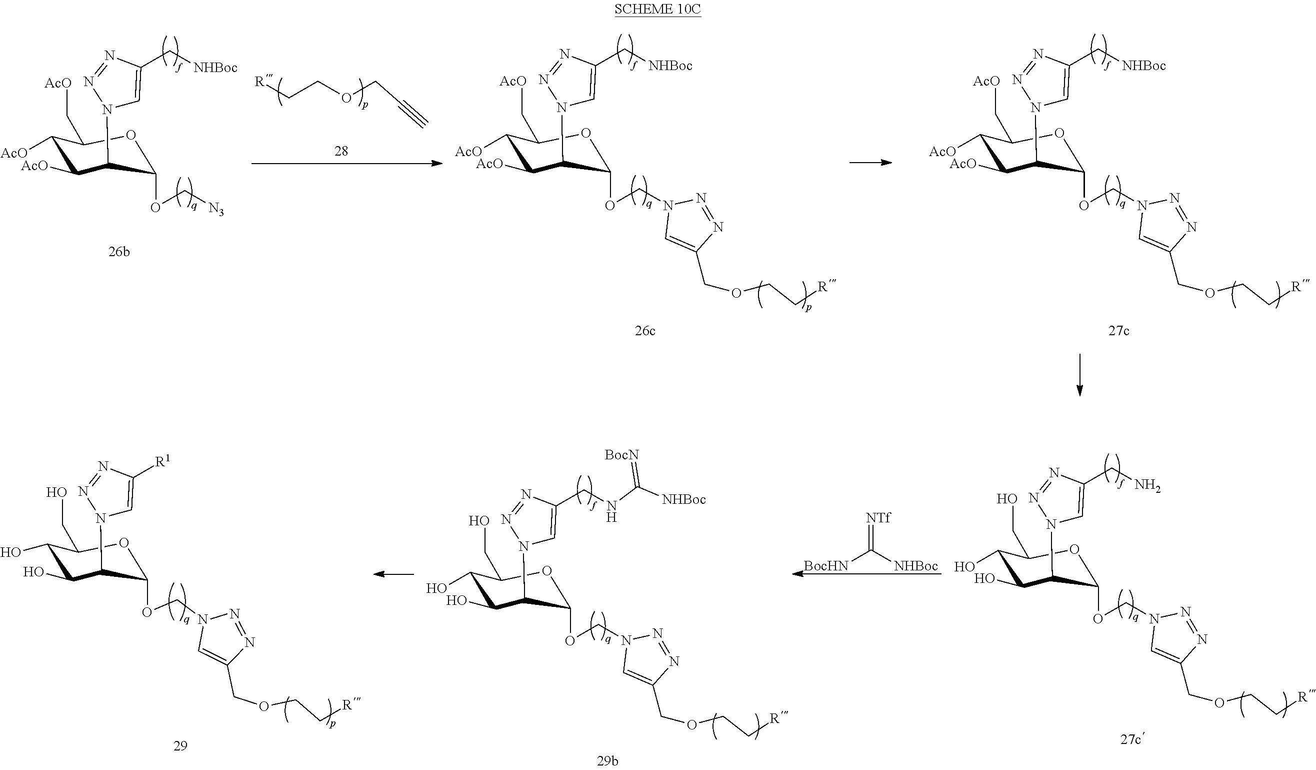

In the example illustrated in the above scheme, the intermediate 26b reacts with alkyne 28 to give the compound 26c, which is deacetylated to give 27c. The latter is subjected to removal of the Boc protecting group with an acid to give compound 27c′, which in turn reacts with 1,3-Di-Boc-2-(trifluoromethylsulfonyl)-guanidine to give compound 29b. The latter is subjected to removal of the Boc protecting group with an acid to give the product 29, or it first reacts with a nucleophilic agent and then is subjected to removal of the Boc protecting group in accordance with SCHEME 4 to give the product 29.

### Method of Synthesis No. 11

A method is described for preparing the compound of formula (IA), or a salt thereof, which comprises a cycloaddition reaction between a terminal alkyne 30 and an azide 31 to give compound 32, as shown in the following reaction scheme (SCHEME 11). The azide 31 can be obtained starting from the respective chloride, as described for the synthesis method no. 10.

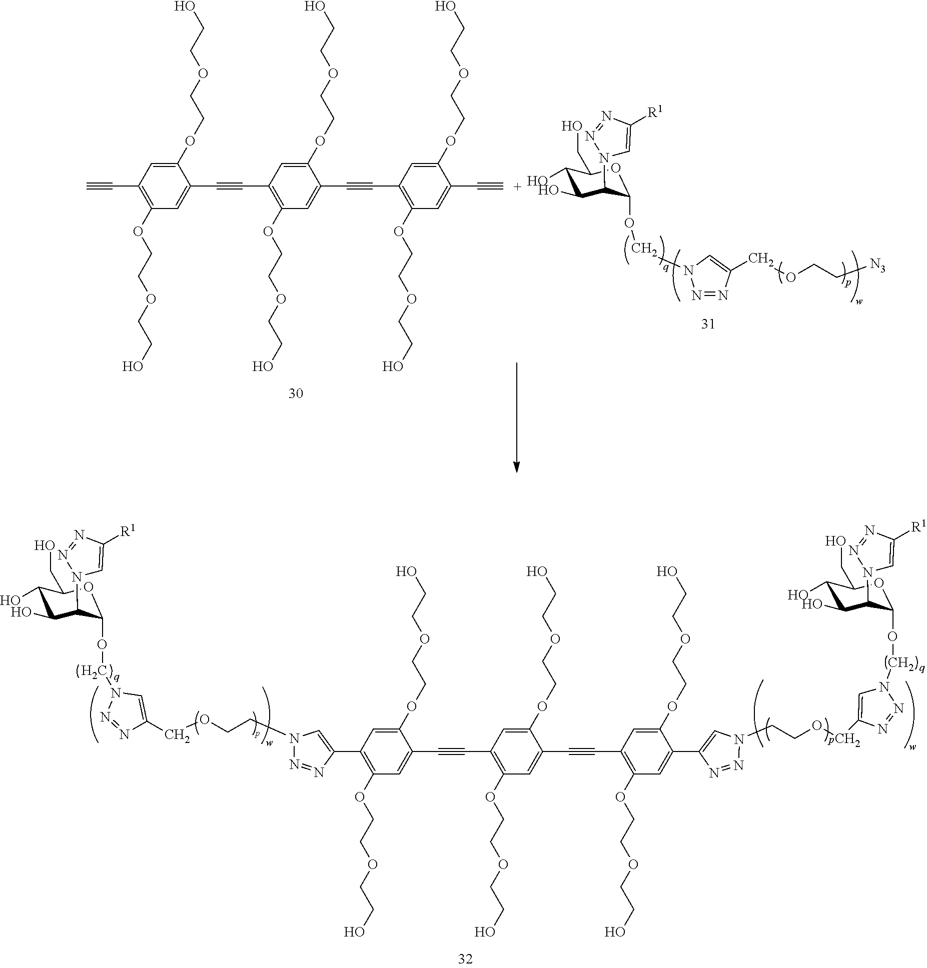

in which:
p is an integer from 0 to 3,
q is an integer from 1 to 3,
w is an integer selected between 0 and 1.

An experimental procedure for obtaining product 32 in accordance with SCHEME 11 is described below.

A 0.01 M CuSO₄·5H₂O solution and a 0.04 M Na-ascorbate solution are prepared in deoxygenated water. A 0.04 M solution of alkyne 30 (according to Ordanini et al. Chem Comm 2015, 51, 3816), a 0.03 M solution of Tris[(1-benzyl-1H-1,2,3-triazol-4-yl)methyl]amine (TBTA) and a 0.2 M solution of compound 31 are prepared in deoxygenated THF.

To the solution of the alkyne 30 (1 mol eq) are added, in sequence: 0.2 mol eq of the TBTA solution; 0.1 mol eq of the CuSO₄·5H₂O solution; 0.4 mol eq of the Na-ascorbate solution. The mixture is stirred at room temperature for 10 minutes, then 2.2 mol eq of the solution of compound 31 are added. The reaction mixture is stirred under MW irradiation at 60° C. At the end of the reaction (monitoring by TLC), the solvents are evaporated and the crude product is purified by automated reverse phase flash chromatography with obtaining the pure product 32 as a yellow foam.

As an alternative to the method according to SCHEME 11, it is possible to prepare compound (IA) starting from a reaction intermediate of compound (I), as will be better described in the experimental part.

### Experimental Part

### Synthesis of Compounds 106a and 106b:

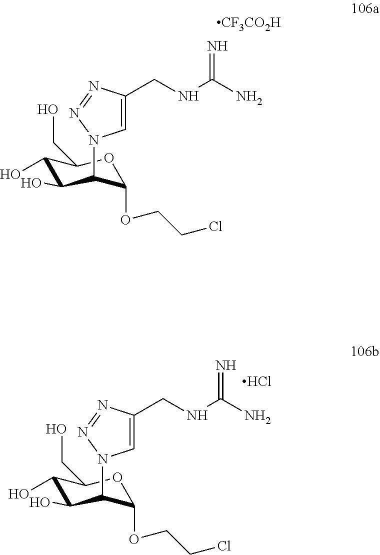

Compounds 106a and 106b were synthesized according to the following reaction scheme:

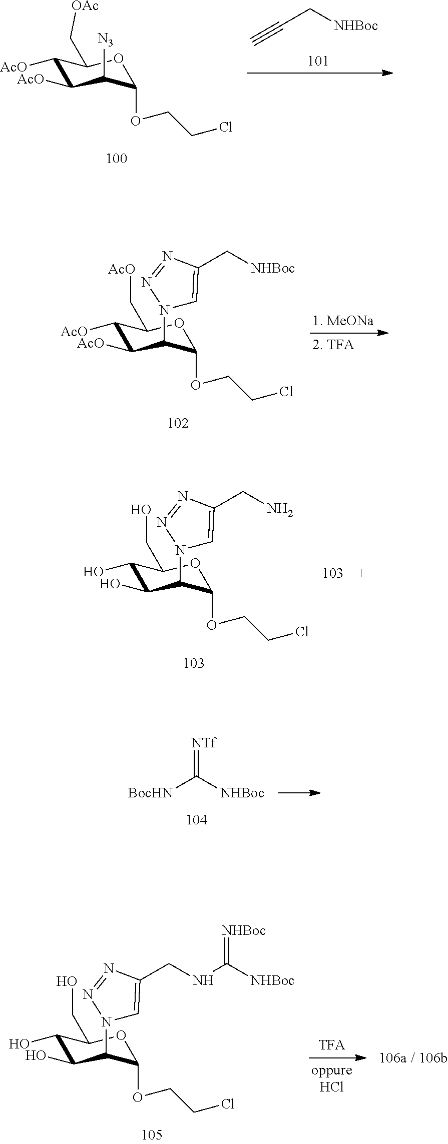

Synthesis of compound 100:

The 2-deoxy-2-azido-1,3,4,6-tetra-O-acetylmannose (prepared according to Medve et al Chem Eur. J. 2019, 14659; 5.6 g, 15.0 mmol, 1 eq.) was dissolved anhydrous CH₂Cl₂ (17 mL) and 2-chloroethanol (2 mL, 30.0 mmol, 2 eq.). The mixture was cooled to 0° C., then BF₃·Et₂O (7.4 mL, 60 mmol, 4 eq.) was added slowly. The reaction was stirred at room temperature, under N₂ for one night. Upon completion (TLC monitoring, toluene: AcOEt 7:3, staining by ceric ammonium molybdate reagent), the mixture was extracted with cold H₂O (x2), saturated solution of NaHCO₃ (x1) and cold H₂O (x1). The organic phase was dried over Na₂SO₄, filtered and concentrated under vacuum. The crude product was purified by automated flash chromatography (toluene with AcOEt gradient from 0% to 30%) to obtain the pure product 100 as a white foam (3.82 g, 65%).

¹H-NMR (400 MHz, CDCl₃): δ(ppm)=5.39 (dd, 1H, J₃₋₄=9.7 Hz, J₂₋₃=3.8 Hz, H₃), 5.32 (t, 1H, J₄₋₃=J₄₋₅=9.7 Hz, H₄), 4.89 (d, 1H, J₁₋₂=1.3 Hz, H₁), 4.23 (dd, 1H, J_{6a-6b}=12.3 Hz, J₆ₐ₋₅=4.9 Hz, H₆ₐ), 4.12-4.04 (m, 2H, H₅+H_{6b}), 3.96-3.89 (m, 1H, Hd7a), 3.84-3.76 (m, 1H, H_{7b}), 3.67 (t, 2H, J₈₋₇=5.5 Hz, H₈), 2.10 (s, 6H, 2x OAc), 2.05 (s, 3H, OAc)

¹³C-NMR (400 MHz, CDCl₃): δ(ppm)=170.8 (CH₃CO), 170.0 (CH₃CO), 169.7 (CH₃CO), 98.7 (C₁), 71.0 (C₃), 68.9 (C₅), 65.6 (C₇), 65.9 (C₄), 62.2 (C₆), 61.5 (C₂), 42.6 (C₈), 20.8 (CH₃CO), 20.7 (CH₃CO), 20.6 (CH₃CO)

MS (ESI): m/z calculated for [C₁₄H₂₀ClN₃NaO₈]⁺: 416.08 [M+Na]⁺, found: 416.26.[α]_{D}¹⁵=+68(c=2.0, CHCl₃)

### Synthesis of Compound 103:

A 0.04 M CuSO₄·5H₂O solution and a 0.16 M Na-ascorbate solution were prepared in deoxygenated water. A 0.4 M solution of N-Boc-propargylamine 101 and a 0.4 M solution of azide 100 were prepared in deoxygenated THF. To the solution of 101 (3.2 mL, 1.27 mmol, 1 mol eq), the following were added in sequence: 0.1 mol eq of the CuSO₄·5H₂O solution (3.2 mL, 0.127 mmol), 0.4 mol eq of the Na-ascorbate solution (3.2 mL, 0.51 mmol), 1 mol eq of the solution of 100 (3.2 mL, 1.27 mmol). The reaction was stirred at room temperature, under N₂ and protected from light. Upon completion (TLC monitoring Hex: AcOEt 6:4, R_{f}: 0.27), the solvents were evaporated. Crude product 102 was purified by automated flash chromatography (hexane with AcoEt gradient from 50% to 100%) to obtain a white foam (yield: 94%; R_{f}: 0.21 in Hex: AcoEt 1:1). Product 102 (50 mg) was dissolved in anhydrous MeOH (1 mL), a freshly prepared 0.1 M NaOMe solution in anhydrous MeOH (200 μL) was then added and the reaction stirred at room temperature for one hour. Upon completion (TLC monitoring CH₂Cl₂:MeOH 9:1), the reaction was neutralized with Amberlite® IR120 hydrogen form ion-exchange resin, filtered and concentrated under vacuum. The crude was dissolved in a 4:1 mixture of anhydrous CH₂Cl₂ and TFA (2.3 mL). The reaction was stirred at room temperature, under N₂ for one hour. Upon completion (TLC monitoring, CH₂Cl₂:MeOH 8:2), the mixture was concentrated under vacuum and co-evaporated with toluene three times to obtain the pure product 103 as TFA salt (white foam; 82% on two steps).

### Synthesis of Compound 105:

The TFA salt of 103 (38 mg, 0.087 mmol, 1 eq.) Was dissolved in H₂O (725 μL) and diluted with 1,4-dioxane (3.6 mL), then 1 was added, 3-Di-Boc-2-(trifluoromethylsulfonyl) guanidine 104 (40.9 mg, 0.1 mmol, 1.2 eq). After five minutes, triethylamine (36 μL, 0.26 mmol, 3 eq) was slowly added and the reaction stirred at room temperature overnight. Upon completion (TLC monitoring, CH₂Cl₂:MeOH 9:1), the mixture was diluted with AcOEt and washed three times with brine. The organic phase was dried over Na₂SO₄, filtered and concentrated under vacuum. The crude was purified by automated flash chromatography (CH₂Cl₂ with MeOH gradient from 0% to 20%) to obtain the pure product 105 as a white foam (30 mg, 61%).

¹H-NMR (400 MHz, CD₃OD): δ(ppm)=8.17 (s, 1H, H_{TrCH}), 5.14 (s, 1H, H₁), 5.09 (dd, 1H, J₂₋₃=5.3 Hz, J₂₋₁=1.1 Hz, H₂), 4.65 (s, 2H, H₉), 4.19 (dd, 1H, J₃₋₄=9.1 Hz, J₃₋₂=5.3 Hz, H₃), 4.04 (m, 1H, H₇ₐ), 3.92-3.77 (m, 4H, H₆+H_{7b}+H₅), 3.76-3.67 (m, 3H, H₈+H₄), 1.52 (s, 9H, 3xCH₃), 1.48 (s, 9H, 3xCH₃).

MS (ESI): m/z calculated for [C₂₂H₃₈ClN₆O₉]⁺: 565.24 [M+H]⁺, found: 565.20; m/z calculated for [C₂₂H₃₇ClN₆NaO₉]⁺: 587.22 [M+Na]⁺, found: 587.21; m/z calculated for [C₂₂H₃₆ClN₆O₉]⁻: 563.22 [M−H]⁻, found: 563.17

### Synthesis of Compound 106a:

Compound 105 (47.4 mg, 0.083 mmol, 1 eq.) Was dissolved in a 4:1 mixture of anhydrous CH₂Cl₂ and TFA (1.66 mL). The reaction was stirred at room temperature, under N₂ for 5 hours. Upon completion (TLC monitoring, CH₂Cl₂:MeOH 8:2), the mixture was concentrated in vacuo and co-evaporated with toluene three times to obtain the pure product 106a as a white foam (39 mg, 98%).

¹H-NMR (400 MHz, CD₃OD): δ(ppm)=8.22 (s, 1H, H_{TrCH}), 5.15-5.08 (m, 2H, H₁+H₂), 4.49 (s, 2H, H₉), 4.22 (dd, 1H, J₃₋₄=9.2 Hz, J₃₋₂=5.1 Hz, H₃), 4.04 (m, 1H, H₇ₐ), 3.88 (d, 2H, J₆₋₅=3 Hz, H₆), 3.86-3.78 (m, 2H, H_{7b}+H₅), 3.77-3.69 (m, 3H, H₈+H₄).

¹³C-NMR (400 MHz, CD₃OD): δ(ppm)=158.9 (C₁₀), 158.8 (C_{Trq}), 124.9 (C_{TrCH}), 99.4 (C₁), 75.1 (C₅), 70.1 (C₃), 69.3 (C₇), 67.6 (C₄), 65.4 (C₂), 61.8 (C₆), 43.8 (C₈), 37.6 (C₉).

MS (HRMS) m/z calculated for [C₁₂H₂₂ClN₆O₅]⁺: 365.1340 [M+H]⁺, found: 365.1337; m/z calculated for [C₁₂H₂₁ClN₆NaO₅]⁺387.1159 [M+Na]⁺, found: 387.1153. [α]_{D}²⁷=+10 (c=0.5, MeOH)

### Synthesis of Compound 106b:

Compound 105 (36.3 mg, 0.064 mmol, 1 eq.) was dissolved in CH₂Cl₂ (1.4 mL), then 190 μL of a 1M aqueous solution of HCl (3 eq) was added. The reaction was stirred at room temperature for 3 days. Upon completion (TLC monitoring, CH₂Cl₂:MeOH 8:2), the mixture was concentrated in vacuo and co-evaporated with toluene three times to obtain the pure product 106b as a white foam (22.7 mg, 88%).

An alternative synthesis of intermediate 105 is reported below, according to the following reaction scheme:

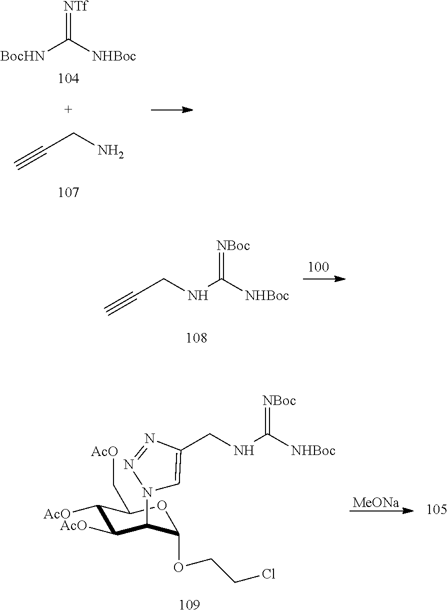

### Synthesis of Compound 108:

1,3-Di-Boc-2-(trifluoromethylsulfonyl)guanidine 104 (160 mg, 0.41 mmol, 0.9 eq) was dissolved in anhydrous dichloromethane (2.05 mL), then triethylamine (63 μL, 0.45 mmol, 1 eq) and propargylamine 107 (29 μL, 0.45 mmol, 1 eq). The reaction was stirred at room temperature, under nitrogen. Upon completion (TLC monitoring Hex:AcOEt 7:3), the mixture was concentrated in vacuo and purified by automated flash chromatography (hexane with AcOEt gradient from 0% to 30%) to obtain compound 108 as a white solid (89.3 mg, 73% product).

¹H-NMR (400 MHz, CDCl₃): δ(ppm)=11.43 (s, 1H, H₃), 8.43 (br s, 1H, H₄), 4.21 (dd, 2H, J₂₋₃=4.9 Hz, J₂₋₁=2.5 Hz, H₂), 2.25 (t, 1H, J₁₋₂=2.5 Hz, H₁), 1.47 (s, 18H, 6xCH₃)

MS (ESI): m/z calculated for [C₁₄H₂₃N₃NaO₄]⁺320.16 [M+Na]⁺, found: 320.02; m/z calculated for [C₁₄H₂₂N₃O₄]⁻: 290.16 [M−H]⁻, found: 295.99

### Synthesis of Compound 109:

A 0.04 M CuSO₄·5H₂O solution and a 0.16 M Na-ascorbate solution were prepared in deoxygenated water. A 0.6 M solution of 108 and a 0.4 M solution of 100 were prepared in deoxygenated THF. To the solution of 108 (375 μL, 0.23 mmol, 1.5 mol eq) were added, in sequence: 0.1 mol eq of the CuSO₄·5H₂O solution (375 μL, 0.015 mmol), 0.4 mol eq of Na-ascorbate solution (375 μL, 0.06 mmol), 1 mol eq of the solution of 100 (375 μL, 0.15 mmol). The reaction was stirred at room temperature, under N₂ and protected from light. Upon completion (TLC monitoring Hex:AcOEt 6:4, Rf: 0.27), the solvents were evaporated and the crude product was purified by automated flash chromatography (hexane with AcOEt gradient from 0% to 70%) to obtain the pure triazole 109 as a white foam (96.7 mg, 93%).

¹H-NMR (400 MHz, CDCl₃): δ(ppm)=11.44 (s, 1H, H₁₁), 8.82 (t, 1H, J₁₀₋₉=4.8 Hz, H₁₀), 7.98 (s, 1H. H_{TrCH}), 5.51 (dd, 1H, J₃₋₄=9.8 Hz, J₃₋₂=5.2 Hz, H₃), 5.41 (dd, 1H, J₂₋₃=5.2 Hz, J₂₋₁=0.9 Hz, H₂), 5.26 (t, 1H, J₄₋₃=9.8 Hz, H₄), 5.14 (s, 1H, H₁), 4.82 (dd, 1H, J_{9a-9b}=15.4 Hz, J₉ₐ₋₁₀=5.6 Hz, H₉a), 4.68 (dd, 1H, J_{9b-9a}=15.4 Hz, J_{9b-10}=5.6 Hz, H_{9b}), 4.33-4.17 (m, 3H, H₆+H₅), 4.02-3.95 (m, 1H, H₇ₐ), 3.90-3.83 (m, 1H, H_{7b}), 3.72 (t, 2H, J₈₋₇=5.7 Hz, H₈), 2.16 (s, 3H, OAc), 2.04 (s, 3H, OAc), 1.95 (s, 3H, OAc), 1.50 (s, 9H, 3xCH₃), 1.48 (s, 9H, 3xCH₃).

MS (ESI): m/z calculated for [C₂₈H₄₄ClN₆O₁₂]⁺: 691.27 [M+H]⁺, found: 691.58; m/z calculated for [C₂₈H₄₃ClN₆NaO₁₂]⁺: 713.25 [M+Na]⁺, found: 713.54; m/z calculated for [C₂₈H₄₂ClN₆O₂₂]⁻: 689.25 [M−H]⁻, found: 689.65

### Synthesis of Compound 105:

To a solution of 109 (50 mg, 0.07 mmol) in anhydrous MeOH (1.2 mL) a freshly prepared 0.1 M NaOMe solution in anhydrous MeOH (200 μL) was added to a final concentration of 0.05 M of the substrate (final NaOMe concentration of 0.014 M). The reaction was stirred at room temperature, under N₂ for one hour. Upon completion (TLC CH₂Cl₂:MeOH 9:1 monitoring), the reaction was neutralized with Amberlite® IR120 hydrogen form ion-exchange resin, filtered and concentrated under vacuum to obtain the pure product 105 as a white foam (37 mg, 94%).

### Synthesis of Compound 113:

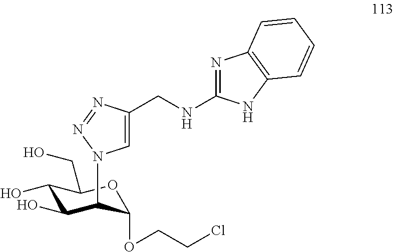

Compound 113 was synthesized according to the following reaction scheme:

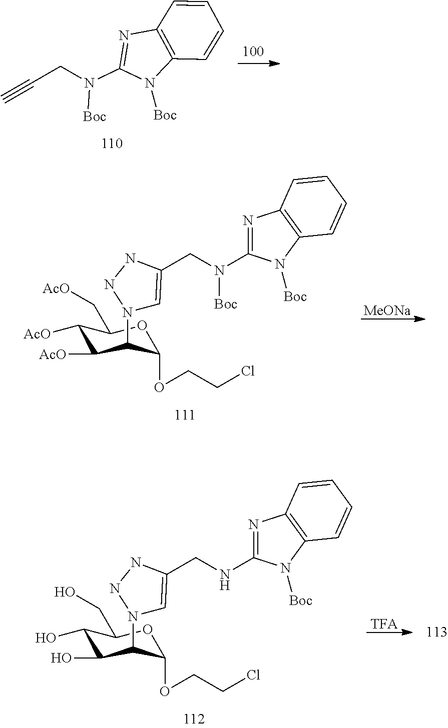

### Synthesis of Compound 111:

A 0.04 M CuSO₄·5H₂O solution and a 0.16 M Na-ascorbate solution were prepared in deoxygenated water. A 0.4 M solution of 110 and a 0.4 M solution of 100 were prepared in deoxygenated THF. To the solution of 110 (800 μL, 0.32 mmol, 1 mol eq) were added, in sequence: 0.1 mol eq of CuSO₄·5H₂O solution (800 μL, 0.032 mmol), 0.4 mol eq of Na-ascorbate solution (800 μL, 0.128 mmol), 1 mol eq of the solution of 100 (800 μL, 0.32 mmol). The reaction was stirred at room temperature, under N₂ and protected from light. Upon completion (TLC Hex:AcOEt 1:1, R_{f}(111): 0.20), the solvents were evaporated, and the crude product purified via automated flash chromatography (Hex with AcOEt gradient from 15% to 100%) to obtain the pure triazole 111 as a white foam (146 mg, 60%).

¹H-NMR (400 MHz, MeOD): δ(ppm)=8.28 (m, 1H, H_{TrCH}), 7.94 (m, 1H, H₁₀), 7.64 (d, 1H, J₁₂₋₁₃=7.1 Hz, H₁₃), 7.37 (m, 2H, H₁₁, H₁₂), 5.46 (m, 1H, H₃), 5.39 (m, 1H, H₂), 5.29 (m, 1H, H₄), 5.18 (m, 1H, H₁), 5.11 (m, 2H, H₉), 4.31 (m, 2H, H_{6a,b}), 4.20 (m, 1H, H₅), 4.05-3.96 (m, 1H, H₇ₐ), 3.92-3.84 (m, 1H, H_{7b}), 3.79 (t, 2H, J₈₋₇=6.6 Hz, H₈), 2.03 (s, 3H, OAc), 1.85 (s, 3H, OAc), 1.50 (s, 3H, OAc), 1.71 (s, 9H, 3xCH₃), 1.40 (s, 9H, 3x CH₃).

### Synthesis of Compound 112:

The product 111 (100 mg) was dissolved in in dry MeOH (720 μL), a freshly prepared 1M NaOMe solution in dry MeOH was added (39 μL) and the reaction was stirred at room temperature for 2 h. Upon completion (TLC CH₂Cl₂:MeOH 9:1), the reaction was neutralized with Amberlite® IR120 hydrogen form ion-exchange resin, filtered and concentrated in vacuo to obtain the pure product 112 as a white foam (66 mg, 94%).

¹H-NMR (400 MHz, CD₃OD): δ(ppm)=8.12 (s, 1H, H_{TrCH}), 7.47 (m, 2H, H_{10,13}), 7.15 (dd, 1H, J₁₁₋₁₂=6.1 Hz, J₁₁₋₁₃=2.8 Hz, H_{11,12}), 5.34 (s, 2H, H₉), 5.08 (s, 1H, H₁), 5.05 (d, J₁₋₂=5.2 Hz, 1H, H₂), 4.15 (dd, 1H, J₃₋₂=9.7 Hz, J₃₋₄=4.0 Hz, H₃), 4.03-3.95 (m, 1H, H₇ₐ), 3.85-3.70 (m, 6H, H₅+H_{6a,6b}+H_{7b}+H_{8a,8b}) 3.64 (t, 1H, H₄), 1.54 (s, 9H, 3x CH₃).

### Synthesis of Compound 113:

Compound 112 (66 mg, 0.12 mmol, 1 eq.) was dissolved in a 4:1 mixture of dry CH₂Cl₂ and TFA (2.45 mL). The reaction was stirred at room temperature, under N₂ for 5 h. Upon completion (TLC monitoring, DCM:MeOH 8:2) the mixture was concentrated in vacuo and co-evaporated with toluene three-times to obtain 113 as the TFA salt (white foam) (66.2 mg, 99%).

¹H-NMR (400 MHz, CD₃OD): δ(ppm)=8.29 (s, 1H, H_{CHTr}), 7.43-7.36 (m, 2H, H_{10,13}), 7.34-7.27 (m, 2H, H_{11,12}), 5.13 (m, 2H, H₁+H₂), 4.74 (s, 2H, H_{9a,9b}), 4.22 (dd, 1H, J₃₋₂=9.3 Hz, J₃₋₄=4.1 Hz, H₃), 4.05-3.97 (m, 1H, H₇ₐ), 3.87 (d, 2H, J₆₋₅=2.9 Hz, H_{6a,6b}), 3.82-3.78 (m, 2H, H_{7b}+H₅), 3.77-3.70 (m, 3H, H_{8a,8b}+H₄).

¹³C-NMR (400 MHz, CD₃OD): δ(ppm)=151.8 (C), 144.3 (C), 131.2 (C_{Trq}), 125.3 (C_{TrCH}), 125.2 (C_{11,12}), 112.6 (C_{10,13}), 99.8 (C₁), 75.2 (C₅), 70.3 (C₃), 69.5 (C₇), 67.7 (C₄), 65.7 (C₂), 61.9 (C₆), 44.1 (C₈), 39.4 (C₉).

MS (ESI) m/z calculated for [C₁₈H₂₄ClN₆O₅]⁺439.87 [M+H]⁺, found: 439.41; m/z calculated for [C₁H₂₄ClN₆O₅]⁻: 437.87 [M−H]⁻, found: 437.34.

### Synthesis of Compound 115:

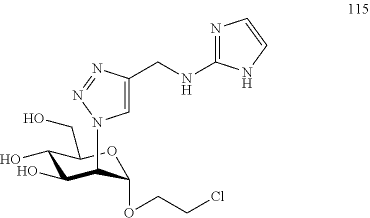

Compound 115 was synthesized according to the following reaction scheme:

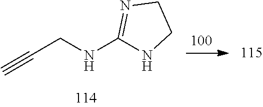

### Synthesis of Compound 115:

A 0.04 M CuSO₄·5H₂O solution and a 0.16 M Na-ascorbate solution were prepared in deoxygenated water. A 0.4 M solution of 114 and a 0.4 M solution of 100 were prepared in deoxygenated THF. To the solution of 114 (1.63 mL, 0.65 mmol, 1 mol eq) were added, in sequence: 0.1 mol eq of CuSO₄·5H₂O solution (1.63 mL, 0.065 mmol), 0.4 mol eq of Na-ascorbate solution (1.63 mL, 0.26 mmol), 1 mol eq of the solution of 100 (1.63 mL, 0.65 mmol). The reaction was stirred at room temperature, under N₂ and protected from light. The reaction was monitored by NMR analysis. After 22 h, the solvents were evaporated, the crude was dissolved in AcOEt and washed two times with NaOH 40% (% m/V). The organic phase was then treated with HCl 1M (until pH=1) and the solvents (AcOEt and water) were evaporated. The obtained chloride salt was purified via semi-preparative HPLC (H₂O with CH₃CN gradient from 0% to 50%+0.1% Formic Acid) to give the pure formate 115 as a white foam (142 mg, 50%).

¹H-NMR (400 MHz, CD₃OD): δ(ppm)=8.21 (s, 1H, H_{CHTr}), 5.12 (s, 2H, H₁+H₂), 4.50 (s, 2H, H_{9a,b}), 4.22 (dd, 1H, J₃₋₂=9.6 Hz, J₃₋₄=3.8 Hz, H₃), 4.01 (m, 1H, H₇), 3.88 (d, 2H, J₆₋₅=4.0 Hz, H_{6,6′}), 3.86-3.71 (m, 9H, H_{7′}+H₅+H₄+H_{8,8′}+H_{10,11}).

¹³C-NMR (400 MHz, CD₃OD): δ(ppm)=161.7 (C), 144.5 (C_{Trq}), 125.0 (C_{TrCH}), 99.8 (C₁), 75.4 (C₅), 70.8 (C₃), 69.7 (C₇), 67.9 (C₅), 66.2 (C₂), 62.2 (C₆), 44.9-43.9 (C₈+C₁₀+C₁₁), 39.0 (C₉).

MS (ESI): m/z calculated for [C₁₄H₂₄ClN₆O₅]+: 390.14 [M+H]⁺, found: 390.27; m/z calculated for [C₁₄H₂₂ClN₆O₅]⁻: 388.14 [M−H]⁻, found: 388.21.

### Synthesis of Compound 119:

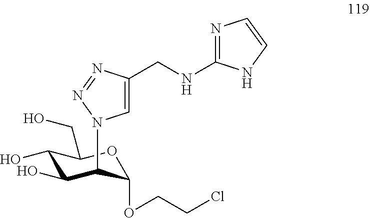

Compound 119 was synthesized according to the following reaction scheme:

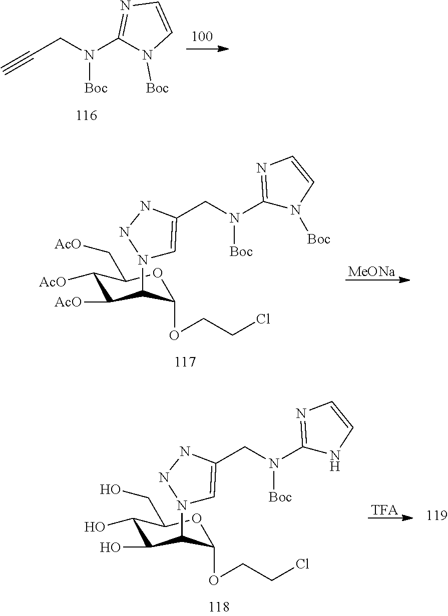

### Synthesis of Compound 117:

A 0.04 M CuSO₄·5H₂O solution and a 0.16 M Na-ascorbate solution were prepared in deoxygenated water. A 0.4 M solution of 116 and a 0.4 M solution of 100 were prepared in deoxygenated THF. To the solution of 116 (850 μL, 0.34 mmol, 1 mol eq) were added, in sequence: 0.1 mol eq of CuSO₄·5H₂O solution (850 μL, 0.034 mmol), 0.4 mol eq of Na-ascorbate solution (850 μL, 0.136 mmol), 1 mol eq of the solution of 1 (850 μL, 0.34 mmol). The reaction was stirred at room temperature, under N₂ and protected from light. Upon completion (TLC DCM:MeOH 95:5,R_{f}(117): 0.46), the solvents were evaporated, and the crude product purified via automated flash chromatography (DCM with MeOH gradient from 0% to 6%) to obtain the pure triazole 117 as a white foam (164 mg, 68%).

¹H-NMR (400 MHz, MeOD): δ(ppm)=8.24 (m, 1H, H_{TrCH}), 7.45 (m, 1H, H₁₁), 6.89 (m, 1H, H₁₀), 5.46 (m, 1H, H₃), 5.39 (m, 1H, H₂), 5.29 (m, 1H, H₄), 5.25 (m, 1H, H₁), 4.95-4.72 (m, 2H, H₉), 4.31 (m, 1H, H₅+H_{6a,6b}), 4.05-3.91 (m, 1H, H_{7a,b}), 3.82 (t, 2H, J₈₋₇=6.6 Hz, H₈), 2.19 (s, 3H, OAc), 2.05 (s, 3H, OAc), 1.90 (s, 3H, OAc), 1.60 (s, 9H, 3xCH₃), 1.38 (s, 9H, 3x CH₃).

### Synthesis of Compound 118:

The product 117 (146 mg) was dissolved in in dry MeOH (4 mL), a freshly prepared 0.1M NaOMe solution in dry MeOH was added (612 μL) and the reaction was stirred at room temperature for 4 h. Upon completion (TLC CHCl₃:MeOH 9:1), the reaction was neutralized with Amberlite® IR120 hydrogen form ion-exchange resin, filtered and concentrated in vacuo to obtain the pure product 118 as a white foam (102 mg, 99%).

¹H-NMR (400 MHz, CDCl₃): δ(ppm)=8.05 (S, 1H, H_{TrCH}), 6.88 (s, 2H, H_{10,11}), 5.20-4.98 (m, 4H, H₁+H₂+H₉), 4.18 (dd, 1H, J₃₋₄=10.0 Hz, J₃₋₂=4.0 Hz, H₃), 4.02 (m, 1H, H₇ₐ), 3.94-3.72 (m, 6H, H₅+H_{6a,b}+H_{7b}+H_{8a,b}), 3.67 (t, 1H, H₄), 1.50 (s, 9H, 3x CH₃).

### Synthesis of Compound 119:

Compound 118 (102 mg, 0.21 mmol, 1 eq.) was dissolved in a 4:1 mixture of dry CH₂Cl₂ and TFA (4.2 mL). The reaction was stirred at room temperature, under N₂ for 3 h. Upon completion (TLC monitoring, DCM:MeOH 8:2) the mixture was concentrated in vacuo and co-evaporated with toluene three-times to obtain 119 (TFA salt) as a white foam (105 mg, 99%).

¹H-NMR (400 MHz, CD₃OD): δ(ppm)=8.22 (s, 1H, H_{CHTr}), 6.88 (s, 2H, H_{10,11}), 5.13-5.10 (m, 2H, H₁+H₂), 4.58 (s, 2H, H₉), 4.21 (dd, J₃₋₂=9.6 Hz, J₃₋₄=4.6 Hz 1H, H₃), 4.01-3.97 (m, 1H, H₇ₐ), 3.90 (d, 2H, J₆₋₅=3.2 Hz, H₆), 3.82-3.76 (m, 2H, H_{7b}+H₅), 3.77-3.69 (m, 2H, H₄+H₈).

¹³C-NMR (400 MHz, CD₃OD): δ(ppm)=144.5 (C), 135.8 (C_{Trq}), 124.7 (C_{TrCH}), 115 (C_{10,11}) 99.5 (C₁), 74.9 (C₅), 70.2 (C₃), 69.4 (C₇), 67.7 (C₄), 65.5 (C₂), 61.9 (C₆), 43.7 (C₈), 39.1 (C₉).

MS (ESI): m/z calculated for [C₁₄H₂₂ClN₆O₅]⁺: 388.73 [M+H]⁺, found: 389.41; m/z calculated for [C₁₄H₂₀ClN₆O₅]⁻: 386.73 [M−H]⁻, found: 386.32.

### Synthesis of Intermediate 121:

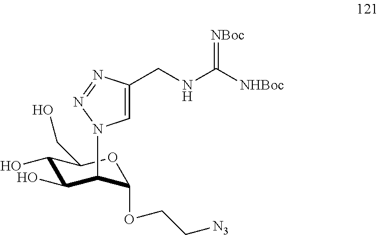

Intermediate 121 was synthesized according to the following reaction scheme:

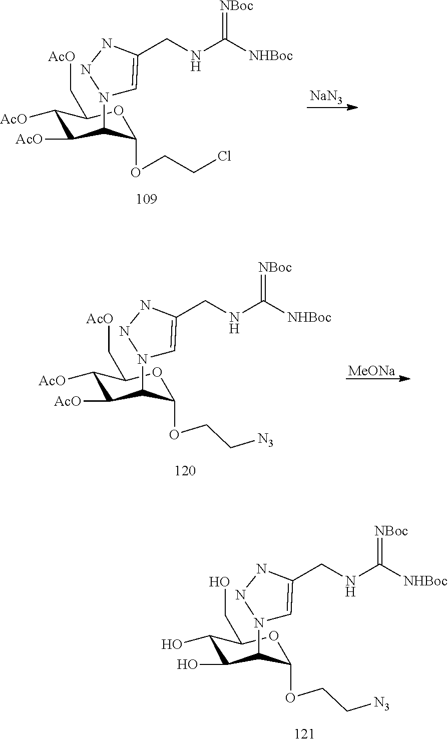

### Synthesis of Compound 120:

Compound 109 (230 mg, 0.33 mmol, 1 eq) was dissolved in anhydrous DMF (1.65 mL), then NaN₃ (106.5 mg, 1.64 mmol, 5 eq) was added and the reaction was stirred at 55° C. for 3 days. Upon completion (¹H-NMR monitoring), the mixture was concentrated in vacuo and the crude product was purified by automated flash chromatography (hexane with AcOEt gradient from 0% to 100%) to obtain the pure product 120 as a white foam. (60.2 mg, 26%).

¹H-NMR (400 MHz, CDCl₃): δ(ppm)=11.44 (s, 1H, H₁₁), 8.82 (t, 1H, J₁₀₋₉=4.6 Hz, H₁₀), 7.98 (s, 1H. H_{TrCH}), 5.49 (dd, 1H, J₃₋₄=9.9 Hz, J₃₋₂=5.2 Hz, H₃), 5.41 (d, 1H, J₂₋₃=5.0 Hz, H₂), 5.26 (t, 1H, J₄₋₃=10.0 Hz, H₄), 5.13 (s, 1H, H₁), 4.80 (dd, 1H, J₉ₐ₋₉b=15.5 Hz, J₉ₐ₋₁₀=5.5 Hz, H₉ₐ), 4.66 (dd, 1H, J_{9b-9a}=15.5 Hz, J_{9b-10}=5.5 Hz, H_{9b}), 4.30 (dd, 1H, J_{6a-6b}=12.4 Hz, J₆ₐ₋₅=4.0 Hz, H₆ₐ), 4.22-4.14 (m, 2H, H_{6b}+H₅), 3.94-3.87 (m, 1H, H₇ₐ), 3.74-3.67 (m, 1H, H_{7b}), 3.58-3.44 (m, 2H, H₈), 2.14 (s, 3H, OAc), 2.02 (s, 3H, OAc), 1.93 (s, 3H, OAc), 1.48 (s, 9H, 3xCH₃), 1.46 (s, 9H, 3x CH₃).

MS (ESI) m/z calculated for [C₂₈H₄₃N₉NaO₁₂]⁺: 720.29 [M+Na]⁺, found: 720.11.

### Synthesis of Compound 121:

To a solution of 120 (50 mg, 0.07 mmol) in anhydrous MeOH (1.2 mL) a freshly prepared 0.1 M NaOMe solution in anhydrous MeOH (200 μL) was added to a final concentration of substrate of 0.05 M (final NaOMe concentration of 0.014 M). The reaction was stirred at room temperature, under N₂ for one hour. Upon completion (TLC CH₂Cl₂:MeOH 9:1 monitoring), the reaction was neutralized with Amberlite® IR120 hydrogen form ion-exchange resin, filtered and concentrated under vacuum to obtain the pure product 121 as a white foam (37 mg, 92%).

An alternative synthesis of intermediate 121 is reported below, according to the following reaction scheme:

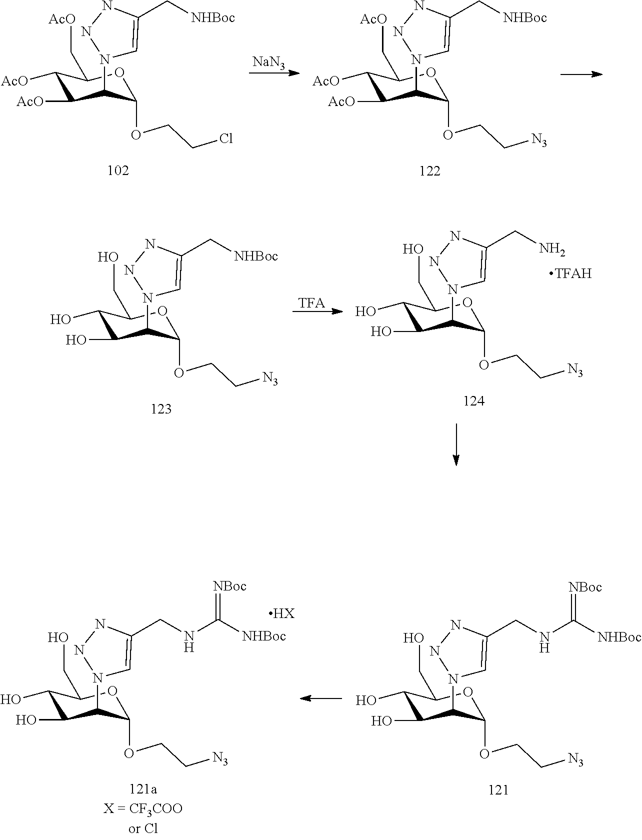

### Synthesis of Compound 122:

Compound 102 (180 mg, 0.33 mmol, 1 eq) was dissolved in anhydrous DMF (1.65 mL), then NaN₃ (106.5 mg, 1.64 mmol, 5 eq) was added and the reaction was stirred at 55° C. for 3 days. Upon completion (¹H-NMR monitoring), the mixture was concentrated in vacuo and the crude product was purified by automated flash chromatography (hexane with 30% to 70% AcOEt gradient) to obtain pure product 122 as a white foam (158 mg, 86%).

¹H-NMR (400 MHz, CD₃OD): δ(ppm)=8.10 (s, 1H, H_{CHTr}), 5.49 (dd, 1H, J₃₋₄=10.2 Hz, J₃₋₂=5.1 Hz, H₃), 5.40 (d, 1H, J₂₋₃=5.1 Hz, H₂), 5.32 (t, 1H, J₄₋₃=10.2 Hz, H₄), 5.27 (s, 1H. H₁), 4.41-4.32 (m, 3H, H₆ₐ+H₉), 4.30-4.19 (m, 2H, H_{6b}+H₅), 4.01-3.92 (m, 1H, H₇ₐ), 3.82-3.75 (m, 1H, H_{7b}), 3.62-3.45 (m, 2H, H₈), 2.16 (s, 3H, OAc), 2.05 (s, 3H, OAc), 1.89 (s, 3H, OAc), 1.45 (s, 9H, 3xCH₃).

MS (ESI): m/z calculated for [C₂₂H₃₃N₇NaO₁₀]⁺: 578.22 [M+Na]⁺, found: 578.38.

### Synthesis of Compound 123:

To a solution of 122 (50 mg, 0.09 mmol) in anhydrous MeOH (1.5 mL) a freshly prepared 0.1 M NaOMe solution in anhydrous MeOH (270 μL) was added to a final concentration of substrate of 0.05 M (final NaOMe concentration of 0.015 M). The reaction was stirred at room temperature, under N₂ for one hour. Upon completion (TLC CH₂Cl₂:MeOH 9:1 monitoring), the reaction was neutralized with Amberlite® IR120 hydrogen form ion-exchange resin, filtered and concentrated under vacuum to obtain pure product 123 as a white foam (37.8 mg, 98%).

¹H-NMR (400 MHz, CD₃OD): δ(ppm)=8.06 (s, 1H, H_{CHTr}), 5.12 (s, 1H, H₁), 5.07 (d, 1H, J₂₋₃=4.7 Hz, H₂), 4.31 (s, 2H, H₉), 4.24-4.17 (m, 1H, H₃), 4.01-3.82 (m, 3H, H₇ₐ+H₆), 3.81-3.66 (m, 3H, H_{7b}+H₄+H₅), 3.48 (t, 2H, J₈₋₇=4.6 Hz, H₈), 1.45 (s, 9H, 3xCH₃).

MS (ESI): m/z calculated for [C₁₆H₂₇N₇NaO₇]⁺452.19 [M+Na]⁺, found: 452.19; m/z calculated for [C₃₂H₅₄N₁₄NaO₁₄]⁺: 881.38 [2M+Na]⁺, found: 881.01; m/z calculated for [C₁₆H₂₆N₇O₇]⁺428.18 [M−H]⁻, found 428.18.

### Synthesis of Compound 124:

Compound 123 (37.5 mg, 0.087 mmol, 1 eq.) was dissolved in a 9:1 mixture of anhydrous CH₂Cl₂ and TFA (1.77 mL). The reaction was stirred at room temperature, under N₂ for an hour.

Upon completion (TLC monitoring, CHCl₃:MeOH 8:2), the mixture was concentrated in vacuo and co-evaporated with toluene three times to obtain 124 as the TFA salt (yellowish foam) (38.5 mg, quantitative).

1H-NMR (400 MHz, CD₃OD): δ(ppm)=8.30 (s, 1H, H_{CHTr}), 5.16-5.11 (m, 2H, H₁+H₂), 4.28-4.21 (m, 3H, H₉+H₃), 4.01-3.93 (m, 1H, H₇ₐ), 3.90 (d, 2H, J₆₋₅=3.4 Hz, H₆), 3.82-3.76 (m, 1H, H₅), 3.75-3.66 (m, 2H, H₄+H_{6b}), 3.53-3.46 (t, 2H, J₈₋₇=4.7 Hz, H₈).

### Synthesis of Compound 121:

The salt of TFA 124 (38 mg, 0.087 mmol, 1 eq.) was dissolved in H₂O (725 μL) and diluted with 1,4-dioxane (3.6 mL), then 1,3-Di-Boc-2-(trifluoromethylsulfonyl)guanidine 104 (40.9 mg, 0.1 mmol, 1.2 eq). After five minutes, triethylamine (36 μL, 0.26 mmol, 3 eq) was slowly added and the reaction stirred at room temperature overnight. Upon completion (TLC monitoring, CH₂Cl₂:MeOH 9:1), the mixture was diluted with AcOEt and washed three times with brine. The organic phase was dried over Na₂SO₄, filtered and concentrated under vacuum. The crude was purified by automated flash chromatography (CH₂Cl₂ with MeOH gradient from 0% to 20%) to obtain the pure product 121 as a white foam (30 mg, 60%).

¹H-NMR (400 MHz, CD₃OD): δ(ppm)=8.18 (s, 1H, H_{TrCH}), 5.14 (s, 1H, H₁), 5.09 (d, 1H, J₂₋₃=5.2 Hz, H₂), 4.65 (s, 2H, H₉), 4.21 (dd, 1H, J₃₋₄=9.1 Hz, J₃₋₂=5.2 Hz, H₃), 3.99-3.91 (m, 1H, H₇ₐ), 3.90-3.83 (m, 2H, H₆), 3.81-3.67 (m, 3H, H₄+H₅+H_{7b}), 3.48 (t, 2H, J₈₋₇=5.1 Hz, H₈), 1.51 (s, 9H, 3xCH₃), 1.48 (s, 9H, 3x CH₃).

Compound 121 was treated with trifluoroacetic acid or HCl, as described above for compound 105, obtaining the compounds collectively referred to as 121a.

### Synthesis of Compound 130

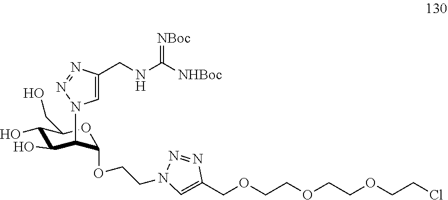

Compound 130 was synthesized according to the following reaction scheme:

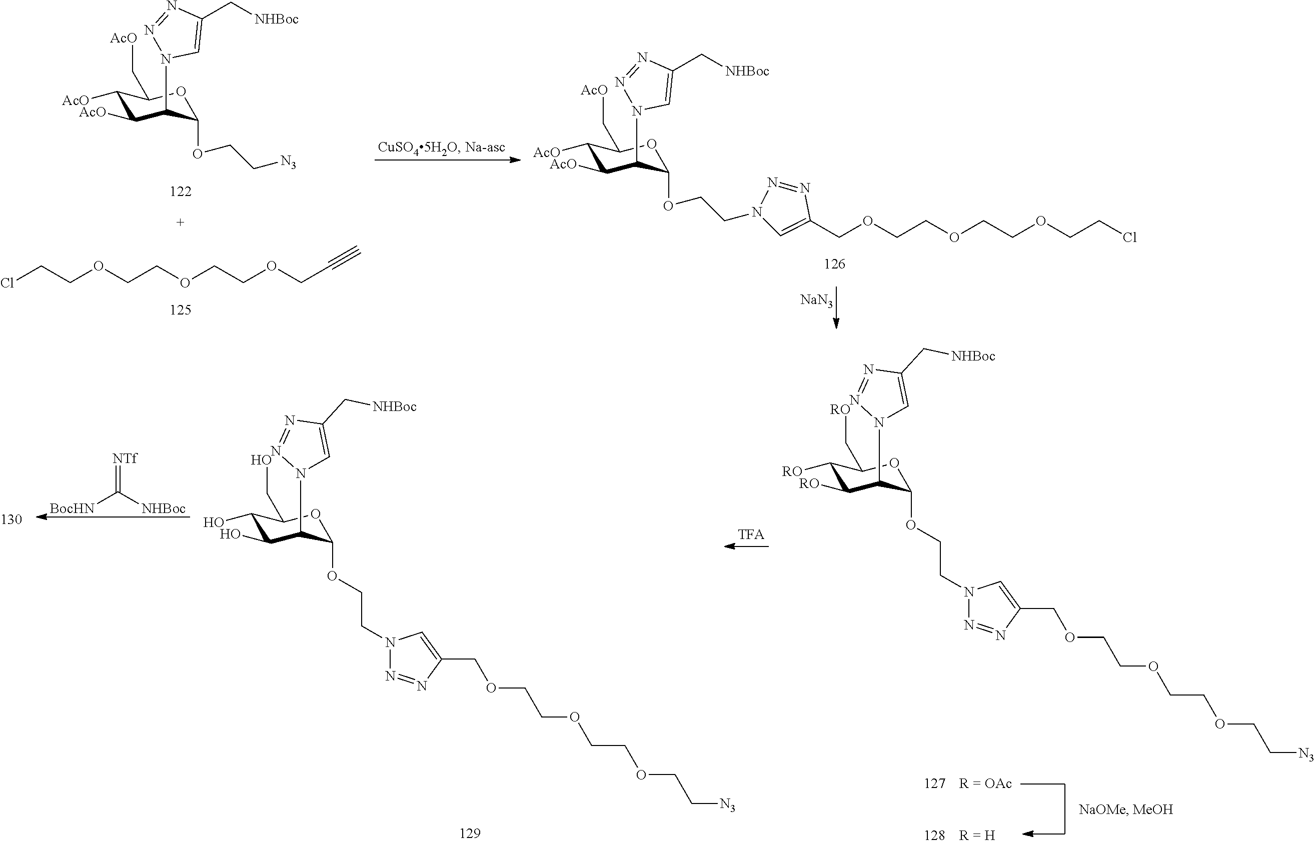

### Synthesis of Compound 126:

A 0.04 M CuSO₄·5H₂O solution and a 0.16 M Na-ascorbate solution were prepared in deoxygenated water. A 0.4 M solution of 125 (prepared according to Piron F. et al, Synthesis (Stuttg) 2010, 2010, 1639-1644) and a 0.4 M solution of 122 were prepared in deoxygenated THF.

To the solution of 125 (2.75 mL, 1.1 mmol, 1 mol eq) were added, in sequence: 0.1 mol eq of the CuSO₄·5H₂O solution (2.75 mL, 0.11 mmol), 0.4 mol eq of the Na-ascorbate solution (2.75 mL, 0.44 mmol) and 1.0 mol eq of the 122 solution (2.75 mL, 1.1 mmol) was added. The reaction was stirred at room temperature in the dark overnight. Upon completion (TLC monitoring CH₂Cl₂:MeOH 9:1), the solvents were evaporated and the crude product purified by automated flash chromatography (CH₂Cl₂ with Acetone gradient from 5% to 100%) to obtain the pure product 126 as a foam (689.4 mg, 82%).

¹H-NMR (400 MHz, CD₃OD): δ(ppm)=8.09 (s, 1H, H_{TrcH}), 8.04 (s, 1H, H_{TrCH}), 5.39-5.29 (m, 2H, H₂, H₃), 5.28-5.17 (m, 2H, H₁, H₄), 4.79-4.70 (m, 2H, H₈), 4.68 (s, 2H, H₁₀), 4.33 (s, 2H, H₉), 4.29-4.16 (m, 2H, H₆ₐ, H₇ₐ), 4.14-3.99 (m, 2H, H_{6b}, H_{7b}), 3.76-3.71 (m, 2H, H₁₅), 3.70-3.62 (m, 11H, H₅, H₁₁—H₁₄, H₁₆), 2.15 (s, 3H, OAc), 2.03 (s, 3H, OAc), 1.88 (s, 3H, OAc), 1.43 (s, 9H, H_{tBu}).

### Synthesis of Compound 127:

Compound 126 (500 mg, 0.65 mmol, 1 eq) was dissolved in dry DMF (3.25 mL), then NaN₃ (213.2 mg, 3.28 mmol, 5 eq) was added, and the reaction stirred at 55° C. for 5 days. Upon completion (¹H-NMR monitoring), the mixture was concentrated in vacuo and the crude product purified via automated flash chromatography (CH₂Cl₂ with MeOH gradient from 3% to 10%) to obtain pure product 127 as a white foam (430 mg, 86%).

¹H-NMR (400 MHz, CD₃OD): δ(ppm)=8.09 (s, 1H, H_{TrCH}), 8.04 (s, 1H, H_{TrCH}), 5.39-5.29 (m, 2H, H₂, H₃), 5.28-5.17 (m, 2H, H₁, H₄), 4.79-4.70 (m, 2H, H₈), 4.68 (s, 2H, H₁₀), 4.33 (s, 2H, H₉), 4.29-4.16 (m, 2H, H₆ₐ, H₇ₐ), 4.14-3.99 (m, 2H, H_{6b}, H_{7b}), 3.72 (m, 11H, H₅, H₁₁-H₁₅), 3.40-3.61 (m, 2H, H₁₆), 2.15 (s, 3H, OAc), 2.03 (s, 3H, OAc), 1.88 (s, 3H, OAc), 1.43 (s, 9H, H_{tBu}).

### Synthesis of Compound 128:

To a solution of 127 (390 mg, 0.51 mmol) in dry MeOH (8.7 mL) a freshly prepared 0.1M NaOMe solution (1.5 mL) in dry MeOH was added to a 0.05M final concentration of the substrate (0.014M final concentration of NaOMe). The reaction was stirred at room temperature, under N₂ for 1 h. Upon completion (TLC monitoring CH₂Cl₂:MeOH 9:1, R_{f}:0.50) the reaction was neutralized with Amberlite® IR120 hydrogen form ion-exchange resin, filtered and concentrated in vacuo to obtain the pure product 128 (311.3 mg, 95%).

¹H-NMR (400 MHz, CD₃OD): δ(ppm)=8.04 (s, 1H, H_{TrCH}), 8.01 (s, 1H, H_{TrCH}), 5.04 (s, 1H, H₁), 4.99 (d, 1H, J₂₋₃=5.1 Hz, H₂), 4.71-4.63 (m, 4H, H₈, H₁₀), 4.29 (s, 2H, H₉), 4.21-4.13 (m, 1H, H₇ₐ), 4.05 (dd, 1H, J₃₋₄=9.4 Hz, J₃₋₂=5.1 Hz, H₃), 3.98-3.91 (m, 1H, H_{7b}), 3.85-3.75 (m, 2H, H₆), 3.70-3.62 (m, 11H, H₄, H₁₁-H₁₅), 3.40-3.33 (m, 3H, H₅, H₁₆), 1.44 (s, 9H, H_{tBu}).

MS (ESI): m/z calculated for [C₂₅H₄₂N₁₀NaO₁₀]⁺: 665.30 [M+Na]⁺, found: 665.58

### Synthesis of Compound 129:

Compound 128 (60 mg, 0.09 mmol, 1 eq.) was dissolved in a 9:1 mixture of dry CH₂Cl₂ and TFA (1.88 mL). The reaction was stirred at room temperature, under N₂ for 2 h. Upon completion (TLC monitoring, CHCl₃:MeOH 8:2, Rf:0.1) the mixture was concentrated in vacuo and co-evaporated with toluene three-times to obtain the pure TFA salt 129 as a white foam (54.9 mg, 93%).

### Synthesis of Compound 130:

The TFA salt 129 (150 mg, 0.23 mmol, 1 eq.) was dissolved in H₂O (1.9 mL) and diluted with 1,4-dioxane (9.6 mL), then 1,3-Di-Boc-2-(trifluoromethylsulfonyl)guanidine (107.3 mg, 0.27 mmol, 1.2 eq) was added. After five minutes, triethylamine (95 μL, 0.69 mmol, 3 eq) was slowly added and the reaction stirred at RT overnight. Upon completion (TLC monitoring, CH₂Cl₂:MeOH 9:1, R_{f}=0.55) the mixture was diluted with AcOEt and washed with brine three-times. The organic phase was dried over Na₂SO₄, filtered and concentrated in vacuo. The crude was purified via automated flash chromatography (CH₂Cl₂ with a MeOH gradient from 0% to 20%) to obtain the pure product 130 as a white foam (128.1 mg, 71%).

¹H-NMR (400 MHz, CD₃OD): δ(ppm)=8.13 (s, 1H, H_{TrCH}), 8.04 (brs, 1H, H_{TrCH}), 5.06 (s, 1H, H₁), 5.01 (dd, 1H, J₂₋₃=5.1 Hz, J₂₋₁=1.2 Hz, H₂), 4.72-4.60 (m, 6H, H₈, H₁₀, H₉), 4.22-4.13 (m, 1H, H₇ₐ), 4.06 (dd, 1H, J₃₋₄=9.7 Hz, J₃₋₂=5.1 Hz, H₃), 3.98-3.90 (m, 1H, H_{7b}), 3.86-3.76 (m, 2H, H₆), 3.70-3.60 (m, 11H, H₄, H₁₁—H₁₅), 3.41-3.31 (m, 3H, H₅, H₁₆), 1.52 (s, 9H, H_{tBu}), 1.48 (s, 9H, H_{tBu}).

¹³C-NMR (400 MHz, CD₃OD): δ(ppm)=164.4 (COOtBu), 157.4 (COOtBu), 153.9 (C₁₇), 146.0 (C_{Trq′}), 144.8 (C_{Trq}), 125.8 (C_{TrCH′}), 124.8 (C_{TrCH}), 99.2 (C₁), 84.5 (C_{quattBu}), 80.5 (C_{quattBu}), 75.2 (C₅), 71.6, 71.5, 71.4 (C₁₂, C₁₃, C₁₄), 71.0 (C₁₅), 70.8 (C₁₁), 70.3 (C₃), 67.7 (C₄), 67.0 (C₇), 65.2 (C₂), 65.0 (C₁₀), 62.2 (C₆), 51.7 (C₁₆), 51.0 (C₈), 36.9 (C₉), 28.6 (tBu), 28.2 (tBu).

MS (ESI): m/z calculated for [C₃₁H₄₃N₁₂O₁₂]₊785.39 [M+H]⁺, found: 785.40; m/z calculated for [C₃₁H₅₂N₁₂NaO₁₂]⁺: 807.37 [M+Na]⁺, found: 807.44.

### Synthesis of Dimeric Compound 133:

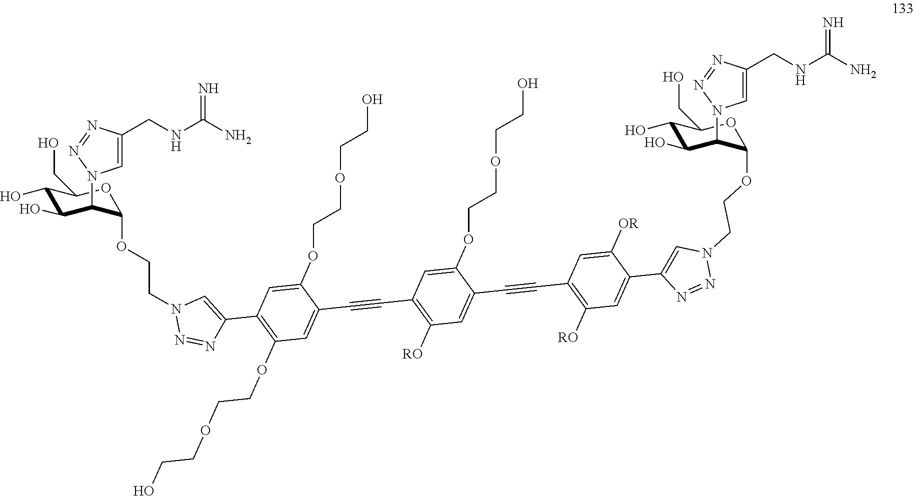

The dimeric compound 133 was synthesized according to the following reaction scheme:

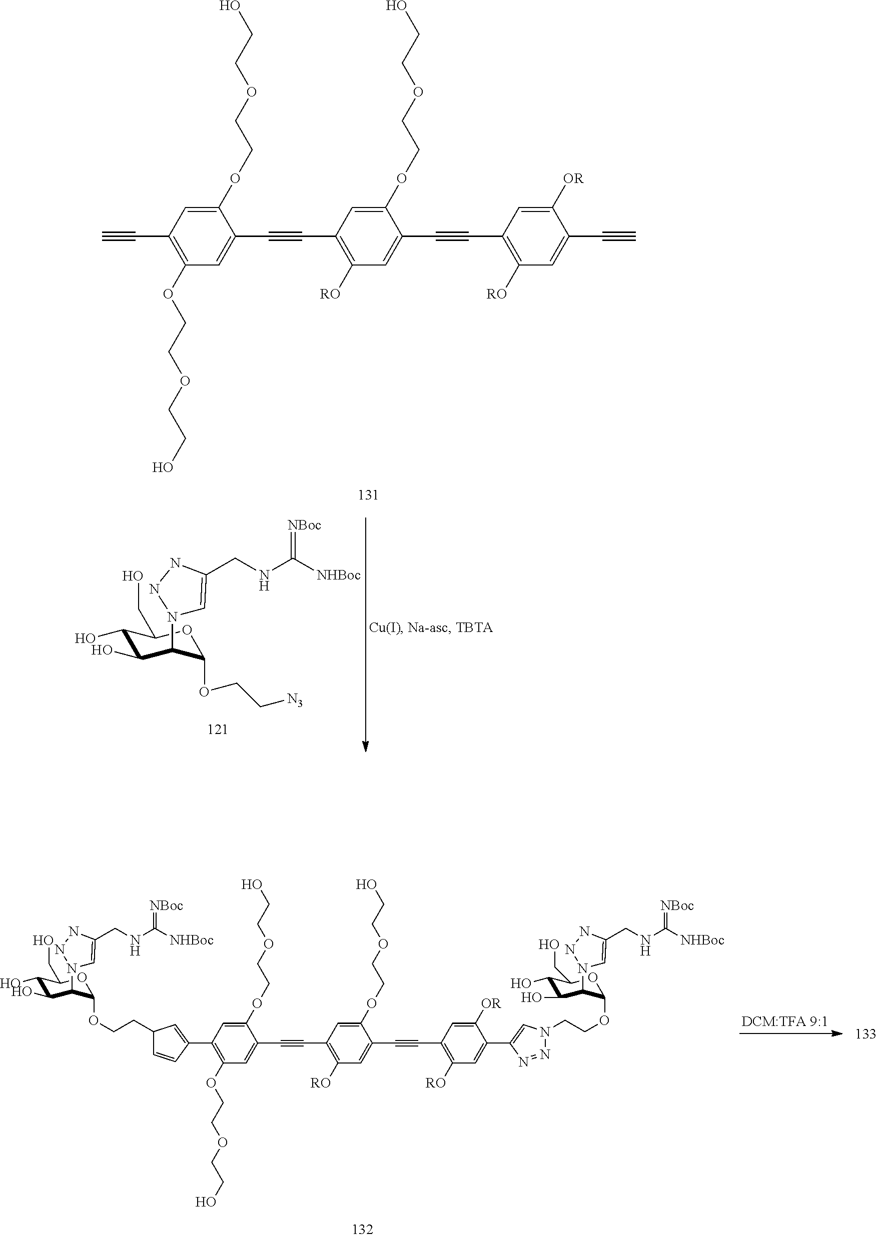

### Synthesis of Compound 132:

A 0.01 M CuSO₄·5H₂O solution and a 0.04 M Na-ascorbate solution were prepared in deoxygenated water. A 0.04 M solution of 131 (prepared according to Ordanini et al. Chem Comm 2015, 51, 3816), a 0.03 M solution of Tris[(1-benzyl-1H-1,2,3-triazol-4-yl)methyl]amine (TBTA) and a 0.2 M solution of 121 were prepared in deoxygenated THF.

To the solution of 131 (600 μL, 24 μmol, 1 mol eq) were added, in sequence: 0.2 mol eq of the TBTA solution (146 μL, 4.4 μmol), 0.1 mol eq of the CuSO₄·5H₂O solution (240 μL, 2.4 μmol) and 0.4 mol eq of the Na-ascorbate solution (240 μL, 9.6 μmol). The mixture was stirred at room temperature for 10 minutes, then 2.2 mol eq of the 121 solution (530 μL, 53 μmol) was added. The reaction was stirred under MW irradiation at 60° C. Upon completion (TLC monitoring H₂O:CH₃CN 1:1), the solvents were evaporated and the crude product was purified via size-exclusion chromatography on a Sephadex LH-20 column (Ø3 cm, H 55 cm; eluent: MeOH) to obtain pure product 132 as a yellow foam (19 mg, 38%)

¹H-NMR (400 MHz, CD₃OD): δ(ppm)=8.61 (s, 2H, H_{T1}), 8.14 (s, 2H, H_{CHtr}), 7.86 (s, 2H, H_{R2}), 7.20 (s, 2H, H_{R5}), 7.15 (s, 2H, HR₁₀), 5.09 (s, 2H, H₁), 5.04 (d, 2H, J₂₋₃=5.1 Hz, H₂), 4.79-4.67 (m, 4H, H₈), 4.67-4.54 (s, 4H, H₉), 4.34-4.30 (m, 4H, H_{G9}), 4.30-4.18 (m, 10H, H_{G1}+H_{G5}+H₇ₛ), 4.11 (dd, 2H, J₃₋₄=9.4 Hz, J₃₋₂=5.3 Hz, H₃), 4.06-3.99 (m, 2H, H_{7b}), 3.98-3.90 (m, 12H, H_{G10}+H_{G2}+H_{G6}), 3.81-3.65 (m, 30H, H₄+H_{G4}+H_{G8}+H_{G12}+H_{G3}+H_{G7}+H_{G11}+H₆), 3.48-3.40 (m, 2H, H₅), 1.50 (s, 18H, 6xCH₃), 1.47 (s, 18H, 6xCH₃).

¹³C-NMR (400 MHz, CD₃OD): δ(ppm)=155.3, 154.9 (C_{R6}, C_{R11}), 154.1 (C₁₀), 150.5 (C_{R3}), 126.9 (C_{TC5}), 122.3 (C_{R1}), 119.1 (C_{R10}), 118.4 (C_{R5}), 115.9, 114.5 (C_{R4}, C_{R9}), 113.0 (C_{R2}), 99.2 (C₁), 92.8, 91.7 (C_{R7}, C_{R8}), 84.6 (C_{quattBu}), 80.6 (C_{quattBu}), 75.3 (C₅), 74.1, 73.7 (C_{G3}, C_{G7}, C_{G11}), 70.9 (C_{G9}, C_{G5} or C_{G1}), 70.8 (C_{G2}, C_{G6}) 70.6 (C_{G10}), 70.3 (C₃), 69.3 (C_{G1} or C_{G5}), 67.7 (C₄), 67.2 (C₇), 65.3 (C₂), 62.4, 62.3, 62.2, 62.1 (C_{G4}, C_{G8}, C_{G12}, C₆), 51.0 (C₈), 37.0 (C₉), 28.6 (tBu), 28.2 (tBu).

MS (MALDI, matrix: DHB, solvent: MeOH): m/z calculated for [C₈₉H₁₂₉N₁₈O₃₄]⁺: 1993.892 [M−1Boc+H]⁺, found: 1994.935; m/z calculated for [C₈₄H₁₂₁N₁₈O₃₂]⁺: 1893.839 [M−2Boc+H]⁺, found: 1894.914; m/z calculated for [C₇₉H₁₁₃N₁₈O₃₀]⁺: 1793.839 [M−3Boc+H]⁺, found: 1794.763; m/z calculated for [C₇₄H₁₀₅N₁₈O₂₈]⁺: 1693.735 [M−4Boc+H]⁺, found: 1694.096.

### Synthesis of Compound 133:

Compound 132 (11 mg, 5 μmol) was dissolved in a 9:1 mixture of anhydrous THF and TFA (900 μL). The reaction was stirred at room temperature, under N₂ for 1 h. Upon completion (¹H-NMR monitoring, CD₃OD), the mixture was concentrated in vacuo and co-evaporated with toluene three times. Crude product was purified via semi-preparative HPLC (conditions reported below) to obtain the pure product 133 (bis-formate salt) as a yellow foam (8.9 mg, 55%).

HPLC: Flow: 10 mL/min; UV channels: 210 nm; 254 nm; A: H₂O+0.1% HCO₂H, B: CH₃CN.
Gradient: 0-5 min: 0% B, 5-20 min: 0-45% B; tᵣ (133)=16.29 min.

¹H-NMR (400 MHz, DMSO-d⁶): δ(ppm)=8.73 (brs, 2H, H₁₀), 8.57 (s, 2H, H_{TC5}), 8.49 (brs, 2H, FA), 8.16 (s, 2H, H_{TrCH}), 7.88-7.75 (m, 8H, H_{R2}, NH, NH₂), 7.22 (s, 2H, H_{R5}), 7.16 (s, 2H, H_{R10}), 5.49 (brs, 2H, OH-3), 5.31 (brs, 2H, OH-4), 5.05 (s, 2H, H₁), 4.94-4.85 (m, 4H, H₂, OH-6), 4.83-4.58 (m, 10H, H₈, OH-Peg), 4.38 (s, 4H, H₉), 4.31-4.19 (m, 12H, H_{G1}, H_{G5}, H_{G9}), 4.12-4.04 (m, 2H, H₇ₐ), 4.00-3.80 (m, 16H, H_{7b}, H₃, H_{G2}, H_{G6}, H_{G10}), 3.71-3.49 (m, 30H, H₆, H₄, H_{G4}, H_{G8}, H_{G12}, H_{G3}, H_{G7}, H_{G11}), 3.44-3.39 (m, 2H, H₅).

¹³C-NMR (400 MHz, CD₃OD): δ(ppm) extrapolated from HSQC=156.9 (FA), 125.2 (C_{TC5}), 123.3 (C_{TrCH}), 117.8 (C_{R10}), 117.5 (C_{R5}), 111.7 (C_{R2}), 97.5 (C₁), 74.8 (C₅), 73.2 (C_{G4}, C_{G8}, C_{G12}, C_{G3}, C_{G7}, C_{G11}), 69.5 (C_{G10}, C_{G2}. C_{G6}), 68.9 (C₃, C_{G9}, C_{G1}, C_{G5}), 66.5 (C₄), 65.8 (C₇), 64.0 (C₂), 60.6 (C₆), 49.9 (C₈), 36.7 (C₉).

HRMS (ESI): m/z calculated for [C₇₄H₁₀₅N₁₈O₂₈]⁺: 1693.7346 [M+H]⁺, found: 1693.7382; m/z calculated for [C₇₄H₁₀₄N₁₈NaO₂₈]⁺: 1715.7165 [M+Na]⁺, found: 1715.7208 (after deconvolution); m/z calculated for [C₇₄H₁₀₆N₁₈O₂₈]²⁺: 847.3712 [M+2H]²⁺, found: 847.3713; m/z calculated for [C₇₄H₁₀₅N₁₈NaO₂₈]²⁺: 858.3622 [M+Na+H]²⁺, found: 858.3618; m/z calculated for [C₇₄H₁₀₄N₁₈Na₂O₂₈]²⁺: 869.3531 [M+2Na]²⁺, found: 869.3525, m/z calculated for [C₇₄H₁₀₆N₁₈NaO₂₈]³⁺: 572.5774 [M+2H+Na]³⁺, found: 572.5781; m/z calculated for [C₇₄H₁₅₆N₁₈Na₂O₂₈]³⁺:579.9047 [M+2Na+H]³⁺, found: 579.9049; m/z calculated for [C₇₄H₁₀₄N₁₈Na₃O₂₈]³⁺: 587.2320 [M+3Na]³⁺, found: 587.2321.

### Synthesis of Dimeric Compound 135:

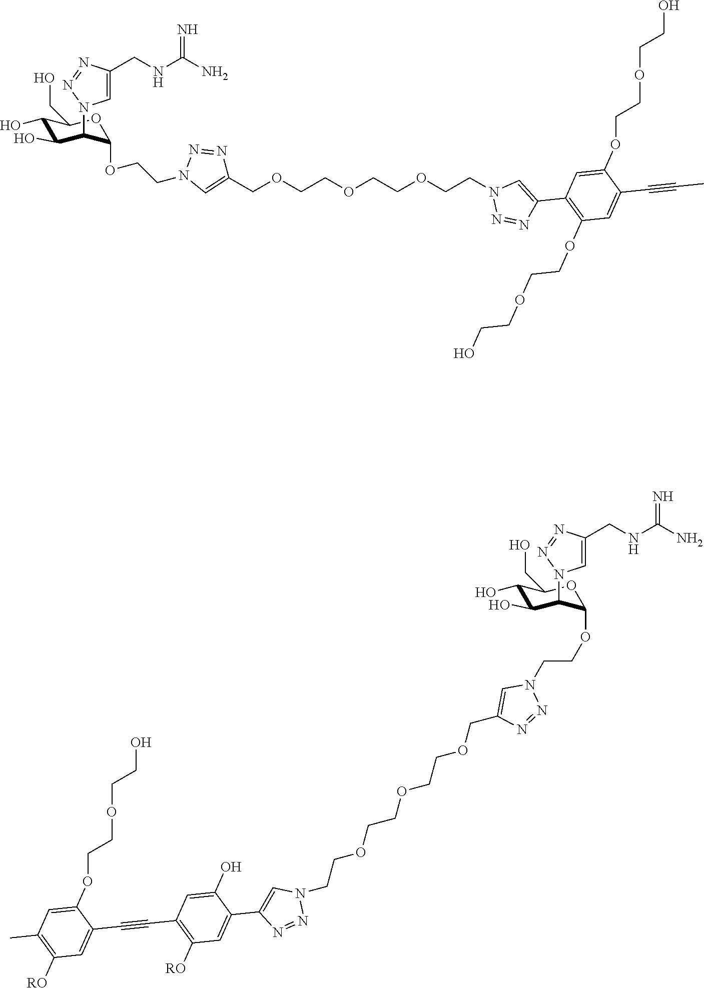

The dimeric compound 135 was synthesized according to the following reaction scheme:

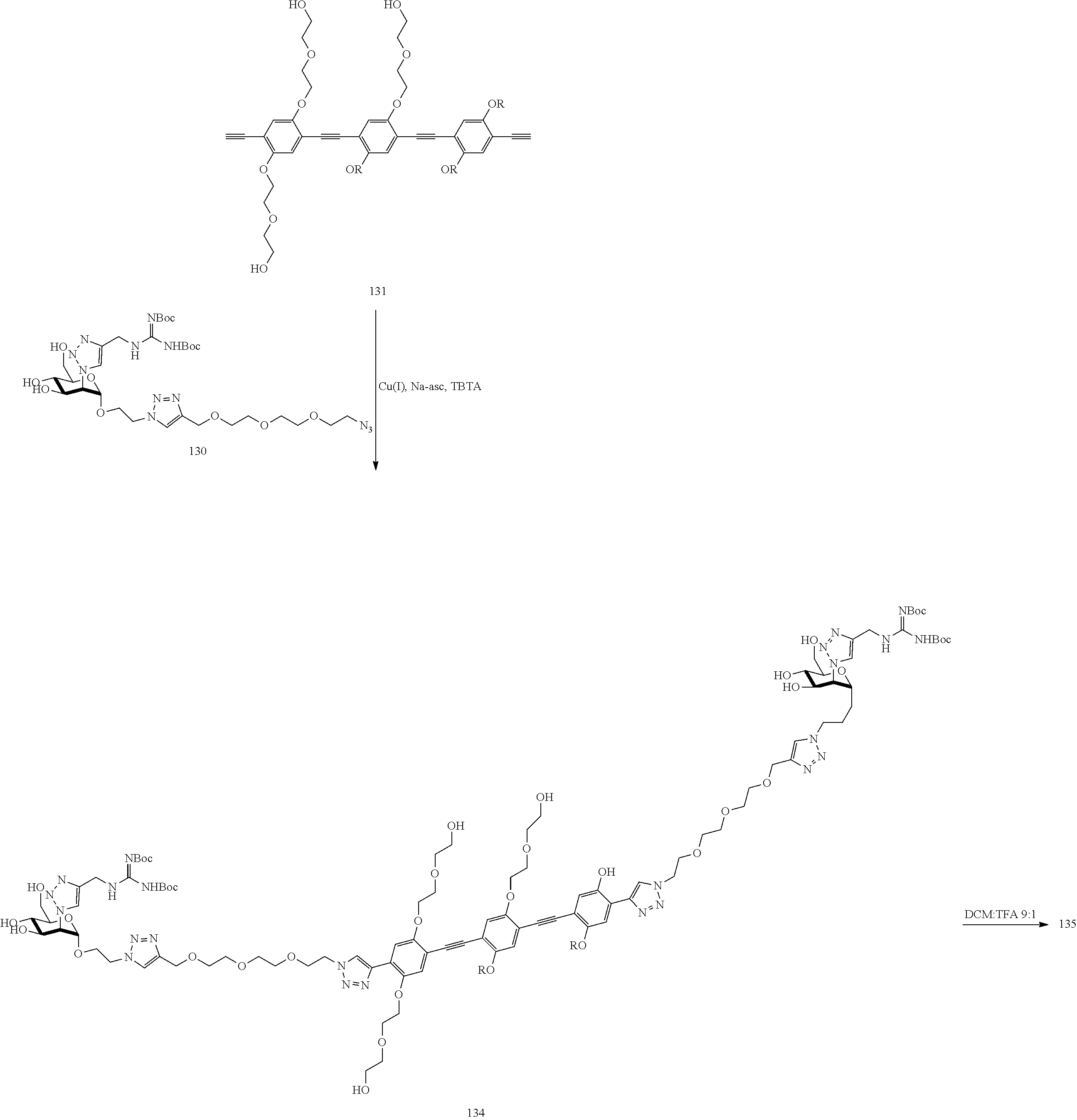

### Synthesis of Compound 134:

A 0.01 M CuSO₄·5H₂O solution and a 0.04 M Na-ascorbate solution were prepared in deoxygenated water. A 0.04 M solution of 131 (prepared according to Ordanini et al. Chem Comm 2015, 51, 3816), a 0.03 M solution of Tris[(1-benzyl-1H-1,2,3-triazol-4-yl)methyl]amine (TBTA) and a 0.2 M solution of 130 were prepared in deoxygenated THF.

To the solution of 131 (1.17 mL, 47 μmol, 1 mol eq) were added, in sequence: 0.2 mol eq of the TBTA solution (313 μL, 9.4 μmol), 0.1 mol eq of the CuSO₄·5H₂O solution (470 μL, 4.7 μmol) and 0.4 mol eq of the Na-ascorbate solution (475 μL, 19 μmol). The mixture was stirred at room temperature for 10 minutes, then 2.2 mol eq of the 130 solution (50 μL, 100 μmol) was added. The reaction was stirred under MW irradiation at 60° C. Upon completion (TLC monitoring H₂O:CH₃CN 1:1), the solvents were evaporated and the crude product was purified via size-exclusion chromatography on a Sephadex LH-20 column (Ø3 cm, H 55 cm; eluent: MeOH) to obtain pure product 134 as a yellow foam (25.5 mg, 22%)

¹H-NMR (400 MHz, CD₃OD): δ(ppm)=8.59 (s, 2H, H_{TC5}), 8.14 (s, 2H, H_{TrCH}), 7.94 (s, 2H, H_{TrCH}), 7.87 (s, 2H, H_{R2}), 7.24 (s, 2H, H_{R5}), 7.19 (s, 2H, H_{R10}), 5.03 (s, 2H, H₁), 4.98 (d, 2H, J₂₋₃=5.2 Hz, H₂), 4.70-4.52 (m, 16H, H₈, H₁₆, H₉, H₁₀), 4.34 (t, 4H, J_{G9-G10}=4.4 Hz, H_{G9}), 4.30-4.24 (m, 8H, H_{G1}, H_{G5}), 4.16-4.12 (m, 2H, H₇ₐ), 4.04 (dd, 2H, J₃₋₄=9.5 Hz, J₃₋₂=5.2 Hz, H₃), 3.98-3.87 (m, 18H, H_{G10}, H_{G2}, H_{G6}, H_{7b}, H₁₅), 3.81-3.54 (m, 46H, H₄, H_{G4}, H_{G8}, H_{G12}, H_{G3}, H_{G7}, H_{G11}, H₆, H₁₁, H₁₂, H₁₃, H₁₄), 1.51 (s, 18H, H_{tBu}), 1.48 (s, 18H, H_{tBu}).

HRMS (ESI) m/z calculated for [C₁₁₂H₁₆₇N₂₄NaO₄₂]⁺: 2520.1670 [M+H]⁺, found: 2520.1633; m/z calculated for [C₁₁₂H₁₆₆N₂₄NaO₄₂]+: 2542.1489 [M+Na]⁺, found: 2542.1438; m/z calculated for [C₁₁₂H₁₅₅N₂₄Na₂O₄₂]²⁺: 2564.1309 [M+2Na—H]⁺, found: 2564.1274 (after deconvolution). m/z calculated for [C₁₁₂H₁₆₈N₂₄O₄₂]²⁺: 1260.5874 [M+2H]²⁺, found: 1260.5823; m/z calculated for [C₁₁₂H₁₆₇N₂₄NaO₄₂]²⁺: 1271.5783 [M+Na+H]²⁺, found: 1271.5763; m/z calculated for [C₁₁₂H₁₆₆N₂₄Na₂O₄₂]²⁺: 1282.5693 [M+2Na]²⁺, found: 1282.5676; m/z calculated for [C₁₁₂H₁₆₉N₂₄O₄₂]³⁺: 840.7275 [M+3H]³⁺, found: 840.7244; m/z calculated for [C₁₁₂H₁₆₈N₂₄NaO₄₂]³⁺: 848.0548 [M+Na+2H]³⁺, found: 848.0525; m/z calculated for [C₁₁₂H₁₆₇N₂₄Na₂O₄₂]⁺855.3821 [M+2Na+H]³⁺, found: 855.3794; m/z calculated for [C₁₁₂H₁₆₆N₂₄Na₃O₄₂]³⁺: 862.7095 [M+3H]³⁺, found: 862.7066.

### Synthesis of Compound 135:

Compound 134 (14.5 mg, 5.7 μmol) was dissolved in a 9:1 mixture of anhydrous CH₂Cl₂ and TFA (570 μL). The reaction was stirred at room temperature, under N₂ for 1 h. Upon completion (¹H-NMR monitoring, CD₃OD), the mixture was concentrated in vacuo and co-evaporated with toluene three times. Crude product was purified via semi-preparative HPLC (conditions reported below) to obtain the pure product 135 as bis-formate salt (4.3 mg, 35%).

HPLC: Flow: 10 mL/min; UV channels: 210 nm; 254 nm; A: H₂O+0.1% HCO₂H, B: CH₃CN.

### Gradient: 0-2 min: 0% B, 2-27 min: 0-40% B; tᵣ (135)=22.04 min

¹H-NMR (400 MHz, DMSO-d⁶): δ(ppm)=8.57 (brs, 2H, H₁₇), 8.50 (s, 2H, H_{TC5}), 8.44 (brs, 2H, FA), 8.14 (s, 2H, H_{TrCH}), 8.09 (s, 2H, H_{TrCH}), 7.86 (s, 2H, H_{R2}), 7.70 (brs, 6H, NH, NH₂), 7.20 (s, 2H, H_{R5}), 7.16 (s, 2H, H_{R10}), 5.47 (brs, 2H, OH-3), 5.26 (brs, 2H, OH-4), 5.01 (s, 2H, H₁), 4.87 (d, 2H, J₂₋₃=4.8 Hz, H₂), 4.73 (brs, 2H, OH-6), 4.68-4.55 (m, 14H, H₈, H₁₆, OH-Peg), 4.51 (s, 4H, H₁₀), 4.39 (s, 4H, H₉), 4.28-4.20 (m, 12H, H_{G1}, H_{G9}, H_{G5}), 4.05-3.98 (m, 2H, H₇ₐ), 3.91-3.80 (m, 20H, H_{7b}, H₃, H₅, H_{G6}, H_{G2}, H_{G10}), 3.61-3.48 (m, 40H, H₆, H₄, H₁₁-H₁₅, H_{G4}, H_{G8}, H_{G12}, H_{G3}, H_{G7}, H_{G11}).

HRMS (ESI): m/z calculated for [C₉₂H₁₃₅N₂₄O₃₄]⁺: 2119.9573 [M+H]⁺, found: 2119.9578; m/z calculated for [C₉₂H₁₃₄N₂₄NaO₃₄]⁺: 2141.9392 [M+H]⁺, found: 2141.9407; m/z calculated for [C₉₂H₁₃₃N₂₄Na₂O₃₄]⁺: 2163.9211 [M+2Na—H]⁺, found: 2163.9216 (after deconvolution); m/z calculated for [C₉₂H₁₃₆N₂₄O₃₄]²⁺: 1060.4825 [M+2H]²⁺, found: 1060.4799; m/z calculated for [C₉₂H₁₃₅N₂₄NaO₃₄]²⁺: 1071.4735 [M+Na+H]²⁺, found: 1071.4708; m/z calculated for [C₉₂H₁₃₄N₂₄Na₂O₃₄]²⁺: 1082.4645 [M+2Na]²⁺, found: 1082.4614; m/z calculated for [C₉₂H₁₃₇N₂₄O₃₄]³⁺: 707.3243 [M+3H]³⁺, found: 707.3232; m/z calculated for [C₉₂H₁₃₆N₂₄NaO₃₄]³⁺: 714.6516 [M+2H+Na]³⁺, found: 714.6503; m/z calculated for [C₉₂H₁₃₅N₂₄Na₂O₃₄]³⁺: 721.9789 [M+2Na+H]³⁺, found: 721.9775; m/z calculated for [C₉₀H₁₃₀N₂₀Na₃O₃₄]³⁺: 729.3062 [M+3Na]³⁺, found: 729.6389.

### Synthesis of Compound 139:

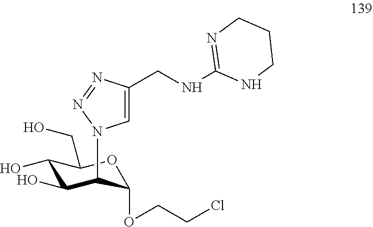

Compound 139 was synthesized according to the following reaction scheme:

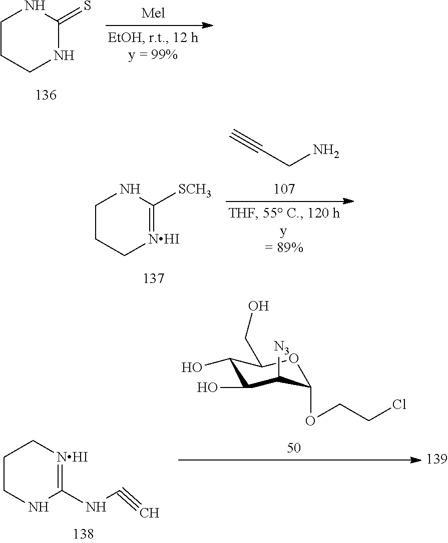

### Synthesis of Compound 137:

Methyl iodide (0.321 mL, 5.16 mmol, 1.2 eq) was added drop by drop during 5 minutes into a stirring suspension of trimethylenthiourea 136 (500 mg, 4.30 mmol) in dry ethanol (4.30 mL) at room temperature. After stirring the reaction mixture at room temperature for 12 hours (¹H-NMR monitoring), the solvent and the excess of methyl iodide were removed under vacuum. The collected residue was washed with Et20, and the precipitate was dried in vacuo for 12 hours at 40° C., to give compound 137 as a white solid (1.09 g, 4.29 mmol, 99%).

¹H NMR (400 MHz, DMSO): δ(ppm)=9.55 (bs, 2H, NH), 3.38 (t, 4H, J_{4/6-5}=5.8 Hz, H₄+H₆), 2.58 (s, 3H, SMe), 1.90 (q, 2H, J_{5-4/6}=5.8 Hz, H₅).

MS (ESI): m/z calculated for [C₅H₁₁N₂S]⁺: 131.06 [M+H]⁺, found: 131.12.

### Synthesis of Compound 138:

Propargylamine 107 (0.149 mL, 2.33 mmol, 1.2 eq) was added while stirring to a suspension of compound 137 (500 mg, 1.94 mmol) in dry THF (1.94 mL) at room temperature. The reaction mixture was stirred at 60° C. for 120 hours and monitored by ¹H-NMR analysis. After total consumption of the starting material, the mixture was evaporated under reduced pressure. The residue was washed with Et₂O and the precipitate was dried in vacuo for 12 hours at 40° C., leading to compound 138 as a light-orange solid (459 mg, 1.73 mmol, 89%).

¹H NMR (400 MHz, CD₃OD): δ(ppm)=3.98 (d, 2H, J₂₋₁=2.5 Hz, H₂), 3.38 (t, 4H, J_{4/6-5}=5.7 Hz, H₄+H₆), 2.83 (t, 1H, J₁₋₂=2.5 Hz, H₁), 1.97 (q, 2H, J_{5-4/6}=5.7 Hz, H₅). ¹³C-NMR (400 MHz, CDCl₃): δ(ppm) extrapolated from HSQC=73.1 (C₁), 38.1 (C₄+C₆), 29.2 (C₂), 20.0 (C₅).

MS (ESI): m/z calculated for [C₇H₁₂N₃]⁺: 138.10 [M+H]⁺, found: 138.07.

### Synthesis of Compound 139:

A 0.4 M alkyne 138 (64 mg, 0.24 mmol, 1 eq.) and a 0.4 M azide 50 (65 mg, 0.24 mmol, 1 eq.) solutions were prepared in deoxygenated HPLC-grade THF. A 0.04 M CuSO₄₋₅H₂O and a 0.16 M Na-ascorbate solutions were prepared in deoxygenated HPLC-grade water. To the alkyne solution (1 eq.) were added: CuSO₄·5H₂O solution (0.1 eq.), Na-ascorbate solution (0.4 eq.) and azide solution (1 eq.). The reaction was stirred at room temperature, under N₂ atmosphere and protected from light. Upon completion (TLC analysis), the solvents were removed under vacuum and the obtained crude was purified via semi-preparative HPLC (H₂O+0.1% HCOOH with CH₃CH gradient (0-5 min: 0%, 5-20:30%, 20-22:100%), t_{R} (139)=12.8 min) to obtain product 139 as its formate salt.

¹H NMR (400 MHz, CD₃OD): δ(ppm)=8.54 (bs, 1H, H_{HCOOH}), 8.20 (s, 1H, H_{CHTr}), 5.11 (m, 2H, H₁+H₂), 4.44 (s, 2H, H₉), 4.21 (dd, 1H, J₃₋₂=9.1 Hz, J₃₋₄=5.0 Hz, H₃), 4.04-3.98 (m, 1H, H₇ₐ), 3.88 (d, 2H, J₆₋₅=3.9 Hz, H₆), 3.86-3.70 (m, 5H, H_{7b}+H₅+H₄+H₈), 3.38 (t, 4H, J_{11/13-12}=5.7 Hz, H₁₁+H₁₃), 1.97 (q, 2H, J_{12-11/13}=5.7 Hz, H₁₂).

¹³C-NMR (400 MHz, CD₃OD): δ(ppm)=170.3 (C—HCOOH), 154.8 (C₁₀), 144.3 (C_{Trq}), 124.7 (C_{TrCH}), 99.5 (C₁), 75.1 (C₅), 70.2 (C₃), 69.4 (C₇), 67.6 (C₄), 65.5 (C₂), 61.8 (C₆), 43.8 (C₈), 39.8 (C₁₂), 37.2 (C₉), 21.1 (C₁₁+C₁₃).

HRMS (ESI): m/z calculated for [C₁₅H₂₆ClN₆O₅]⁺: 405.1653 [M+H]⁺, found: 405.1654.

### Synthesis of Compound 146:

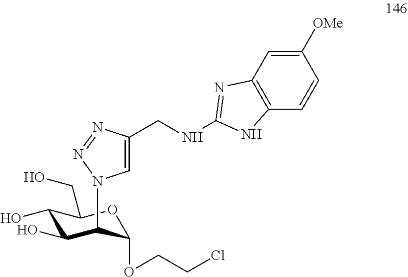

Compound 146 was synthesized according to the following reaction scheme:

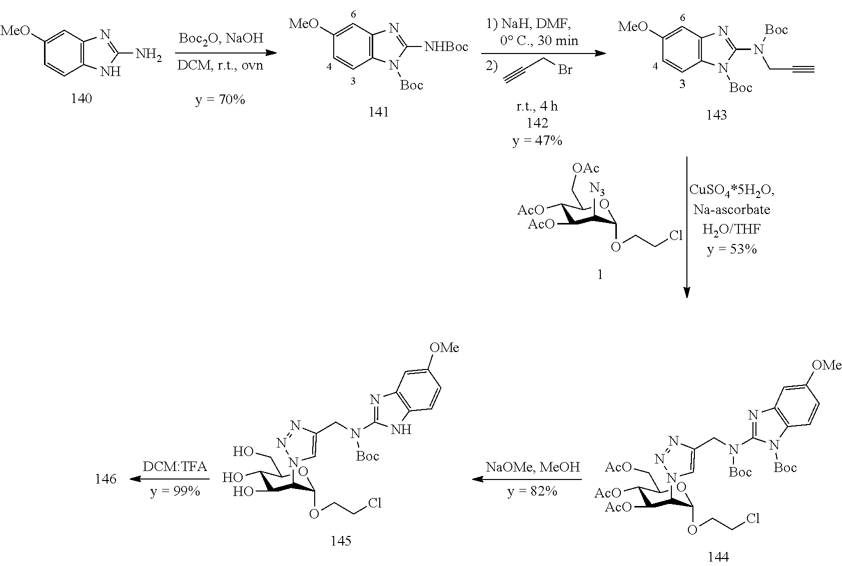

### Synthesis of Compound 141:

2-Amino-5-methoxybenzimidazole 140 (150 mg, 0.90 mmol) was dissolved in 2.7 mL of aqueous NaOH (2M, 2.7 mmol, 3 eq) and stirred at room temperature for 10 minutes. Then, a solution of Boc₂O (393 mg, 1.80 mmol, 2 eq.) in anhydrous CH₂Cl₂ (2.4 mL) was added and the reaction mixture was stirred at room temperature overnight under nitrogen atmosphere. Upon completion of the reaction (TLC monitoring, Hex:AcOEt 7:3, R_{f}=0.23, staining ninhydrine reagent), the two phases were separated. The organic layer was washed with water, dried over anhydrous Na₂SO₄, filtered and concentrated under vacuum. The obtained crude was purified by flash column chromatography (Hex:AcOEt (8:2) to remove apolar impurities and CH₂Cl₂ with Et₂O gradient from 10 to 20% to recover the product), leading to the desired product 141 (mixture of two regioisomers) as a white solid (228 mg, 0.63 mmol, 70%). R_{f} (141): 0.23 in Hex:AcoEt 7:3; R_{f} (141): 0.65 in CH₂Cl₂:Et₂O 9:1.

¹H NMR (400 MHz, CDCl₃, major regioisomer): δ(ppm)=9.78 (bs, 1H, NH), 7.52 (d, 1H, J₃₋₄=8.9 Hz, H₃), 7.22 (d, 1H, J₆₋₄=2.5 Hz, H₆), 6.77 (dd, 1H, J₄₋₃=8.9 Hz, J₄₋₆=2.5 Hz, H₄), 3.82 (s, 3H, OMe), 1.72 (s, 9H, tBu), 1.56 (s, 9H, tBu).

¹H NMR (400 MHz, CDCl₃, minor regioisomer): δ(ppm)=9.69 (bs, 1H, NH), 7.55 (d, 1H, J₃₋₄=8.9 Hz, H₃), 7.26 (m, 1H, H₆), 6.88 (dd, 1H, J₄₋₃=8.9 Hz, J₄₋₆=2.5 Hz, H₄), 3.83 (s, 3H, OMe), 1.74 (s, 9H, tBu), 1.55 (s, 9H, tBu).

MS (ESI): m/z calculated for [C₁₈H₂₅N₃O₅]⁺: 363.18 [M+H]⁺, found: 363.77.

### Synthesis of Compound 143:

NaH (90%, 13 mg, 0.55 mmol, 2eq.) was suspended in anhydrous DMF (0.5 mL) under nitrogen atmosphere, and cooled to 0° C. Then, a solution of 141 (100 mg, 0.28 mmol, 1 eq.) in DMF (1.4 mL) was added, and the reaction mixture was vigorously stirred for 30 minutes at the same temperature. Propargyl bromide 142 (80% in toluene, 48 μL, 0.50 mmol, 1.8 eq.) was added dropwise, warmed to room temperature, and stirred overnight. Upon completion (TLC monitoring, CH₂Cl₂:Et₂O 95:5,R_{f}=0.51), the reaction was quenched with saturated aqueous NaHCO₃ and extracted three times with EtOAc. The combined organic layers were dried over anhydrous Na₂SO₄, filtered, and concentrated under vacuum. The obtained crude was purified by automated flash column chromatography (CH₂Cl₂ with Et₂O gradient from 2 to 10%), leading to the product 143 (mixture of two regioisomers) as a white solid (52 mg, 0.13 mmol, 47%). R_{f} (143): 0.51 in CH₂Cl₂:Et₂O 95:5.

¹H NMR (400 MHz, CDCl₃, major regioisomer) δ (ppm)=7.81 (s, 1H, H₃), 7.21 (d, 1H, J₆₋₄=2.0 Hz, H₆), 6.97 (m, 1H, H₄), 4.66-4.50 (m, 2H, H₂), 3.86 (s, 3H, OMe), 2.18 (s, 1H, H₁), 1.67 (s, 9H, tBu), 1.38 (s, 9H, tBu).

¹³C-NMR (400 MHz, CDCl₃, major regioisomer): δ(ppm) extrapolated from HSQC=116.0 (C3), 114.1 (C4), 102.8 (C6), 72.6 (C1), 55.5 (OMe), 38.1 (C2), 28.4 (tBu).

¹H NMR (400 MHz, CDCl₃, minor regioisomer) δ (ppm)=7.60 (d, 1H, J₃₋₄=9.2 Hz, H₃), 7.55-7.49 (m, 1H, H₆), 6.97 (m, 1H, H₄), 4.66-4.50 (m, 2H, H₂), 3.88 (s, 3H, OMe), 2.18 (s, 1H, H₁), 1.67 (s, 9H, tBu), 1.38 (s, 9H, tBu).

¹³C-NMR (400 MHz, CDCl₃, minor regioisomer): δ(ppm) extrapolated from HSQC=120.2 (C3), 114.1 (C4), 98.9 (C6), 72.6 (C1), 55.5 (OMe), 38.1 (C2), 28.4 (tBu).

MS (ESI): m/z calculated for [C₂₁H₂₇N₃O₅]⁺: 401.20 [M+H]⁺, found: 401.87.

### Synthesis of Compound 144:

A 0.4 M alkyne 143 (52 mg, 0.13 mmol, 1 eq.) and a 0.4 M azide 1 (51 mg, 0.13 mmol, 1 eq.) solutions were prepared in deoxygenated HPLC-grade THF. A 0.04 M CuSO₄·5H₂O and a 0.16 M Na-ascorbate solutions were prepared in deoxygenated HPLC-grade water. To the alkyne solution (1 eq.) were added: CuSO₄·5H₂O solution (0.1 eq.), Na-ascorbate solution (0.4 eq.) and azide solution (1 eq.). The reaction was stirred at room temperature, under N₂ atmosphere and protected from light. Upon completion (TLC analysis), the solvents were removed under vacuum and the obtained crude was purified via flash chromatography (Hex with AcOEt gradient from 40 to 55%) to obtain pure product 144 as a white foam (55 mg, 0.07 mmol, 53%). R_{f} (144): 0.27 in Hex:AcOEt 1:1.

¹H NMR (400 MHz, CD₃OD): δ(ppm)=8.37-8.17 (m, 1H, H_{TrCH}), 7.87-7.43 (m, 1H, H₁₁), 7.23-7.07 (m, 1H, H₁₄), 7.06-6.94 (m, 1H, H₁₂), 5.49-5.23 (m, 3H, H₃+H₂+H₄), 5.19 (s, 1H, H₁), 5.16-4.96 (m, 2H, H₉), 4.37-4.15 (m, 3H, H₅+Hd), 4.06-3.94 (m, 1H, H₇ₐ), 3.93-3.82 (m, 3H, H_{7b}+OMe), 3.79 (t, 2H, J₈₋₇=5.1 Hz, H₈), 2.17-1.29 (m, 30H, OAc (4x)+tBu (2x)).

¹³C-NMR (400 MHz, CD₃OD): δ(ppm) extrapolated from HSQC=124.0 (C_{TrCH}), 115.5 (C₁₁), 113.6 (C₁₂), 102.1 (C₁₄), 97.8 (C₁), 69.8 (C₅), 69.5 (C₇), 69.4 (C₃), 64.6 (C₄), 61.5 (C₆), 60.0 (C₂), 54.8 (OMe), 43.0 (C₉), 41.7 (C₈), 27.4 (tBu), 27.2 (tBu), 20.0 (OAc), 19.9 (OAc), 19.1 (OAc).

### Synthesis of Compound 145:

Compound 144 (25 mg, 0.03 mmol, 1 eq.) was dissolved in dry MeOH (0.6 mL) at room temperature under N₂ atmosphere. A freshly prepared 1 M NaOMe solution in MeOH (10 μL) was slowly dropped to the aforementioned solution. The reaction was stirred at room temperature, under N₂ atmosphere. Upon completion (TLC analysis), the reaction was neutralized with Amberlite® IR120 ion-exchange resin (hydrogen form), filtered and concentrated under vacuum to give the pure product 145 as a white foam (14 mg, 0.02 mmol, 82%). R_{f} (145): 0.48 in CHCl₃:MeOH 9:1.

¹H NMR (400 MHz, CD₃OD): δ (ppm)=8.10 (s, 1H, H_{TrCH}), 7.34 (d, 1H, J₁₁₋₁₂=8.7 Hz, H₁₁), 7.02 (d, 1H, J₁₄₋₁₂=2.4 Hz, H₁₂), 6.79 (dd, 1H, J₁₂₋₁₁=8.7 Hz, J₁₂₋₁₄=2.4 Hz, H₁₂), 5.29 (s, 2H, Hg), 5.08 (d, 1H, J₁₋₂=1.1 Hz, H₁), 5.05 (dd, 1H, J₁₋₂=1.1 Hz, J₂₋₃=5.2 Hz, H₂), 4.15 (dd, 1H, J₃₋₄=9.7 Hz, J₃₋₂=5.2 Hz, H₃), 4.01-3.94 (m, 1H, H₇ₐ), 3.85-3.70 (m, 9H, H₅+H₆+H_{7b}+H₈+OMe), 3.64 (t, 1H, J₄₋₃=9.7 Hz, H₄), 1.53 (s, 9H, tBu).

¹³C-NMR (400 MHz, CD₃OD): δ(ppm) extrapolated from HSQC=123.6 (C_{TrCH}), 110.0 (C₁₂), 102.1 (C₁₁), 98.1 (C₁), 97.0 (C₁₂), 74.0 (C₅), 69.5 (C₃), 68.0 (C₇), 66.9 (C₄), 63.8 (C₂), 61.2 (C₆), 54.7 (OMe), 42.2 (C₈), 41.7 (C₉), 26.4 (tBu).

### Synthesis of Compound 146:

Compound 145 (14 mg, 0.02 mmol, 1 eq.) was dissolved in a 4:1 mixture of dry CH₂Cl₂ and TFA ([Substrate]=0.05 M) and the reaction was stirred at room temperature, under N₂ atmosphere. Upon completion (TLC monitoring), the mixture was concentrated under vacuum and co-evaporated with toluene (3x) to obtain compound 146 as the trifluoroacetate salt (11 mg, 0.02 mmol, 99%). R_{f} (146): 0.74 in CH₂Cl₂: MeOH 8:2.

¹H NMR (400 MHz, CD₃OD): δ(ppm)=8.28 (s, 1H, H_{CHTr}), 7.27 (d, 1H, J₁₁₋₁₂=8.8 Hz, H₁₁), 6.94 (d, 1H, J₁₄₋₁₂=2.2 Hz, H₁₂), 6.88 (dd, 1H, J₁₂₋₁₁=8.8 Hz, J₁₂₋₁₄=2.2 Hz, H₁₂), 5.13-5.11 (m, 2H, H₁+H₂), 4.72 (s, 2H, H₉), 4.21 (dd, 1H, J₃₋₂=9.3 Hz, J₃₋₄=4.8 Hz, H₃), 4.03-3.98 (m, 1H, H₇ₐ), 3.87 (d, 2H, J₆₋₅=3.2 Hz, H₆), 3.85-3.77 (m, 5H, H_{7b}+H₅+OMe), 3.76-3.70 (m, 3H, H₈+H₄).

¹³C-NMR (400 MHz, CD₃OD): δ(ppm)=158.7 (C₁₃), 151.8 (C₁₀) 143.8 (C₁₅+C₁₆), 132.0 (C_{Trq}), 124.9 (C_{TrCH}), 113.0 (C₁₁), 112.1 (C₁₂), 99.5 (C₁), 97.8 (C₁₄), 75.1 (C₅), 70.2 (C₃), 69.4 (C₇), 67.7 (C₄), 65.5 (C₂), 61.9 (C₆), 56.4 (OMe), 43.8 (C₈), 39.2 (C₉).

HRMS (ESI): m/z calculated for [C₁₉H₂₆ClN₆O₆]⁺: 469.1602 [M+H]⁺, found: 469.1608; m/z calculated for [C₁₉H₂₆ClN₆NaO₆]⁺491.1422 [M+Na]⁺, found: 491.1424.

### Experimental Analysis and Results

The inhibitory potency of the monovalent glycomimetic compounds of the invention towards the extra cellular domain (ECD) of the DC-SIGN and L-SIGN receptors was evaluated by competition experiments performed using Surface Plasmon Resonance (SPR) on Biacore T200 instrument at 25° C., using a CM4 sensorchip functionalized with SARS-CoV-2 Spike protein.

The spike protein used was Spike HexaPro, described in Hsiesh et al. Science, 2020, 369, 1501. Its production and purification were performed as described for Spike 2P in Thépaut et al. PLOS Pathogens, 2021, 17, e1009576.

Flow cells Fc1, Fc2 and Fc3 were activated with 60 μL of a mixture of EDC-NHS (according to manufacturer). Fc1 was functionalized with 16.7 μL of a solution of bovine serum albumin (BSA) at 20 μg/mL in 10 mM NaOAc at pH 4 and was used as a reference. Fc2 and Fc3 were functionalized with Spike HexaPro at 20 μg/mL in 10 mM NaOAc pH 5, reaching 1766 and 1913 RU respectively.

The inhibitory potency of the compounds towards DC-SIGN and L-SIGN ECD was evaluated with competition tests performed with decreasing concentrations of the compounds from 5 mM to 9.7656 μM, using a 1:2 dilution. The compounds were co-injected with 20 μM DC-SIGN or 20 μM L-SIGN solutions. The flow was set at 5 μL/min in buffer A (25 mM Tris, 150 mM NaCl, 4 mM CaCl₂) supplemented with 0.05% Tween20, with an association and dissociation time set to 100 seconds each. Surface regeneration was performed for 10 seconds at 100 μL/min with a stabilization period of 100 seconds, using 50 mM EDTA for DC-SIGN tests and 50 mM glycine NaOH pH 12, 0.15% Triton, 25 mM EDTA for L-SIGN tests.

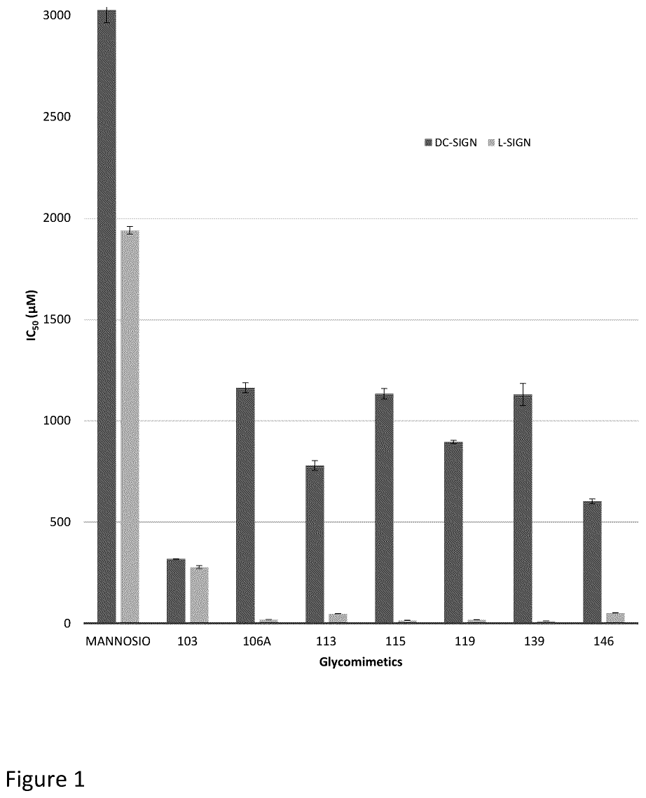

The results of the above competition experiments performed by SPR are illustrated in the graph of FIG. 1, which shows the IC₅₀ of compounds 106a, 113, 115, 119, 139 and 146 (invention) compared with the IC₅₀ of mannose and compound 103 (comparative examples).

From the results reported in FIG. 1 it can be seen that compounds 106a, 113, 115, 119, 139 and 146 are effective and selective L-SIGN binders, with IC50 in the low-medium ρMolar range.

Compound 106a has a selectivity for L-SIGN up to 58 fold over DC-SIGN and, with an IC₅₀ of 24 μM, inhibits the L-SIGN receptor 80 times better than D-mannose (IC₅₀ 1939 μM) and 11 times better than compound 103 (IC₅₀ 278 μM).

Compound 119 (IC₅₀ 18 μM) inhibits the L-SIGN receptor over two orders of magnitude better than D-mannose.

Compounds 113 (IC₅₀=48 μM) and 146 (IC₅₀=52 μM) show a similar affinity for L-SIGN, even though the former is more selective against DC-SIGN.

Compound 139 has a selectivity for L-SIGN up to 94 fold over DC-SIGN and, with an IC₅₀ of 12 μM, is the most potent compound of the series.

Compound 115 is the second most potent inhibitor of L-SIGN in the series, exhibiting a IC₅₀ value of 15 μM and a selectivity for L-SIGN up to 75 fold over DC-SIGN.

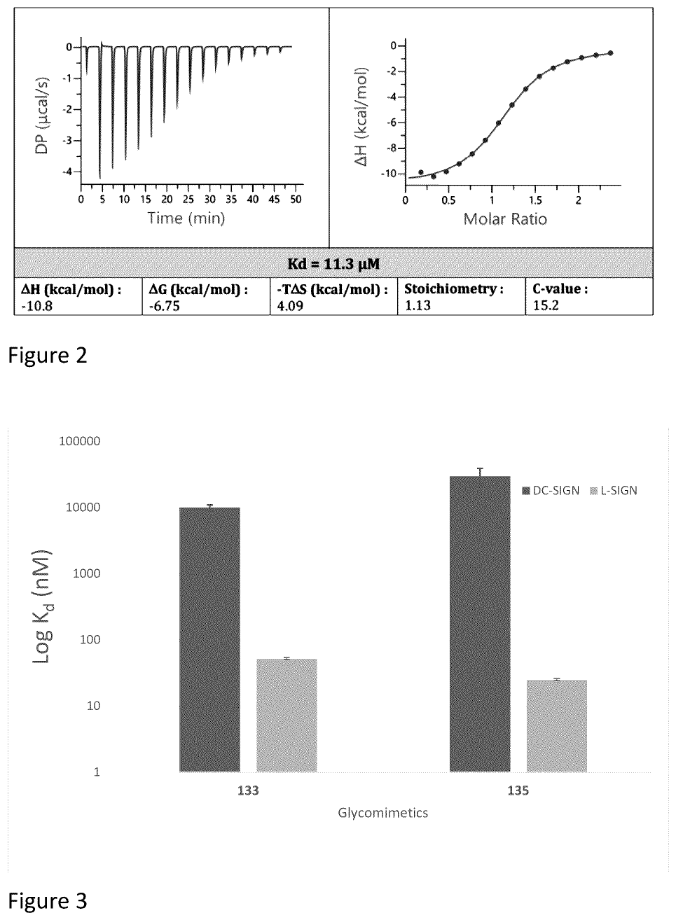

The interaction of compound 106a with the L-SIGN receptor was also confirmed by Isothermal Titration Calorimetry (ITC) experiments (FIG. 2).

These experiments were carried out on a MicroCal PEAQ-ITC apparatus, at 25° C., in buffer A (25 mM Tris pH 8, 150 mM NaCl, 4 mM CaCl₂). L-SIGN ECD (172 μM) was titrated with consecutive 2 μL injections of a 2 mM solution of compound 106a in Calcium buffer (30 μL of 20 mM compound 106a+270 μL of Calcium buffer).

The analysis was performed in triplicate and the graphs shown in FIG. 2 (which report, respectively, Differential Power (DP) vs. time, and ΔH vs. molar ratio) are representative of the average of the three experiments.

The value of the dissociation constant obtained is: Kd=12.8±1.5 μM. This value confirms the high affinity of ligand 106a for the L-SIGN receptor.

The results obtained from the ITC experiments for the ligand 106a, shown in FIG. 2, confirm the excellent value (low micromolar) of the dissociation constant Kd and indicate that the interaction between ligand and L-SIGN receptor has an enthalpy contribution (ΔH) which is very favorable. The stoichiometry of interaction between ligand and L-SIGN receptor is 1,13 and the value of c (C-value) indicates that the concentration of L-SIGN receptor used in this experiment is appropriate for a correct analysis of the data.

The inhibitory potency of the divalent glycomimetic compounds of the invention 133 and 135 towards the extra cellular domain (ECD) of the DC-SIGN and L-SIGN receptors was evaluated by direct interaction experiments performed using Surface Plasmon Resonance (SPR) on Biacore T200 instrument at 25° C., using a CM3 sensor chip S series.

For this experiment the extracellular domain (ECD) of DC-SIGN and L-SIGN were immobilized onto the SPR surface in an oriented manner, by their N-terminus, taking advantage of biotine/streptavidin interaction. To generate DC/L SIGN specifically biotinylated at the N-terminus, 3 glycine residues at the N-terminus of the protein were added by site directed mutagenesis in order to allow a sortaging procedure. The biotinylated versions of DC/L-SIGN ECDs (Biot-ECDs) were prepared by sortaging as described in Achilli, S., Monteiro, J. T., Serna, S., Mayer-Lambertz, S., Thépaut, M., Le Roy, A., Ebel, C., Reichardt, N.-C., Lepenies, B., Fieschi, F., and Vives, C. (2020) TETRALEC, Artificial Tetrameric Lectins: A Tool to Screen Ligand and Pathogen Interactions. IJMS 21, 1-19 using a commercial synthetic biotinylated peptide,

To generate the oriented SPR surfaces, the flow cells were functionalized with Streptavidin in 10 mM Acetate buffer pH 4 and the remaining activated groups were blocked with 1 M ethanolamine pH 8. After blocking, the flow cells were treated with 100 μL of 10 mM HCl to remove non-specifically bound proteins and 100 μL of 50 mM NaOH/1M NaCl to expose surface to regeneration protocol. Priming of the surface was done using the running buffer (25 mM Tris pH 8, 150 mM NaCl, 4 mM CaCl₂), 0.05% Tween). A Streptavidin flow cell surface was used as reference (On Flow Cell 1 of a Biacore T200 with a level of functionalization of 1300 RU) for correction of the binding response. DC-SIGN-Biot-ECD and L-SIGN-Biot_ECD (solubilized in the same running buffer) are injected and captured on FC-2 and FC3 surfaces. When the desired density is reached, injection is stopped. An injection of 12s at 100 μL/min of 1M NaCl/10 mM NaOH was done to eliminate non-specifically captured proteins. For DC-SIGN-Biot_ECD 1547 RU were captured on FC2 and for L-SIGN 1774 RU. Surfaces are ready for interaction. Regeneration of the surfaces was achieved by 50 mM glycine NaOH pH12 0.15% Triton 25 mM EDTA.

Interaction studies and Kd determination were performed by injecting increasing concentrations of the ligands (here 133 and 135) ranging from 2 nM to 500 nM obtained by serial dilution in the running buffer. Binding curves were analyzed using Biacore T200 Evaluation Software 3.2.1 (GE Healthcare) and data were fit using Steady State Affinity model.

The results of the above direct interaction experiments performed by SPR are illustrated in the graph of FIG. 3, which shows the log of the apparent Kd (log Kd, nM) of compounds 133 and 135 (invention) for DC-SIGN and L-SIGN. The data show that these compounds reach nM affinities for L-SIGN (133, Kd 52 nM; 135, Kd 25 nM) and their selectivity over DC-SIGN is 200 fold (133) and 1000-fold (135), respectively.

It is evident that those which have been described are only particular embodiments of the present invention. The person skilled in the art will be able to make to the invention all those modifications necessary for its adaptation to particular conditions, without however departing from the scope of protection as defined in the attached claims.

1. A compound of formula (I) or a salt thereof, 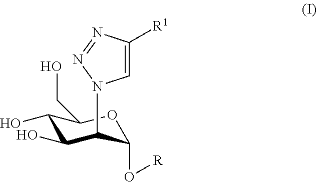
where R is selected from the group consisting of:
C₁-C₆ alkyls, C₃-C₆ cycloalkyls, C₆-C₁₀ aryls, —R²Cl,
—R³NH₂, —R⁴NHC(O) R′, —R⁵SR″, —R⁶OR″, and 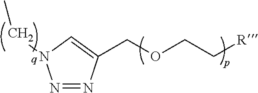
where:
p is an integer between 0 and 3;
q is an integer between 1 and 3;
R², R³, R⁴, R⁵ are divalent radicals selected from the group consisting of C₁-C₆ alkylenes and C₃-C₆ cycloalkylenes; and
R′, R″ are selected from the group consisting of C₁-C₆ alkyls, C₃-C₆ cycloalkyls, C₆-C₁₀ aryls, R″′ is selected from the group consisting of —OH and halogens,
and where R¹ has formula (II): 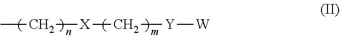
where:
X is selected from the group consisting of CH₂, O, NH and S;
Y is selected from the group consisting of O, NH and S;
n is an integer between 0 and 3;
m is an integer between 0 and 3;
W is a heterocyclic substituent W1 selected from the group consisting of: 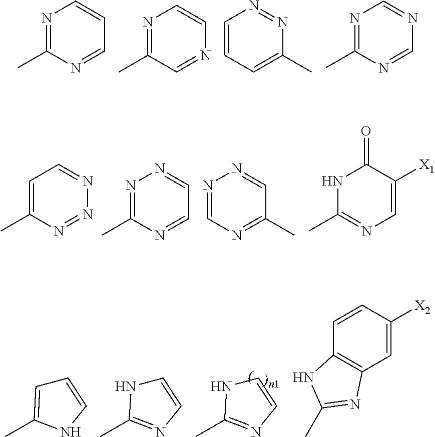
or it is a substituent W2 having general formula 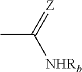
where:
X₁ is selected from the group consisting of H and C₁-C₆ alkyls;
X₂ is selected from the group consisting of H, OCH₃, OH, NH₂, CH₃, CF₃, SH, SR, halogen, where R is as defined above;
n₁ is an integer selected between 1 and 2;
Z is selected from the group consisting of S and NRₐ, in which Rₐ and R_{b} are independently selected from the group consisting of H, COR⁷, COOR⁸, CONHR⁹, where R⁷, R⁸ and R⁹ are H, C₁-C₆ alkyls, C₃-C₆ cycloalkyls, or C₆-C₁₀ aryls;
or compound of formula (IA) or a salt thereof,
A-(B)-C-(B)-A (IA)
in which: 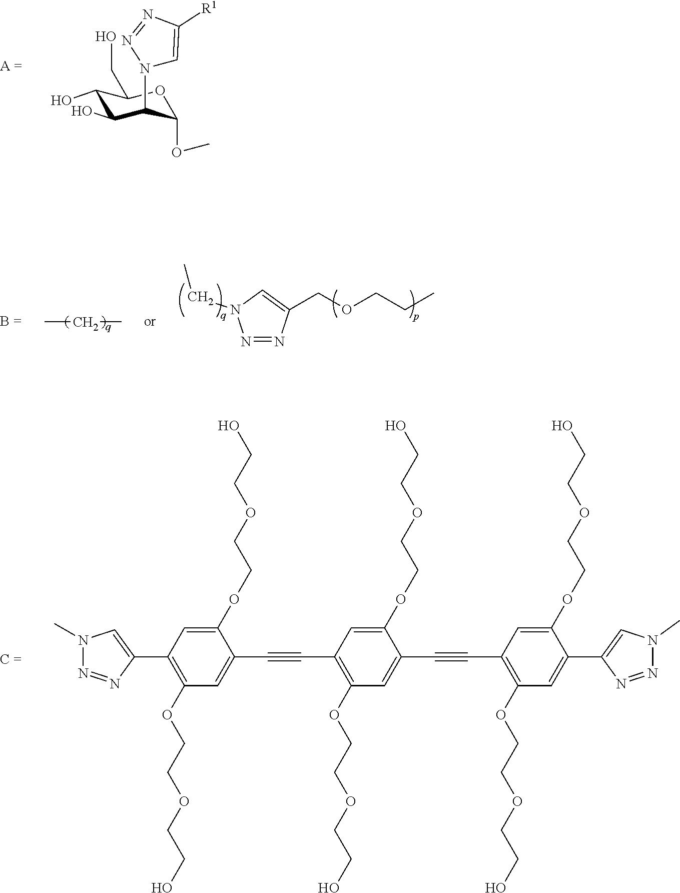
where R′, p and q are as defined above.

2. The compound according to claim 1, wherein R is —CH₂CH₂Cl.

3. The compound according to claim 1, wherein R′ has formula (II-A) 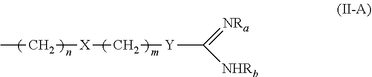
where when n=0, X is CH₂.

4. The compound according to claim 3, wherein:
X═CH₂ and Y is selected from NH, S and O,
n=0 and/or m=0,
Rₐ═H and/or R_{b}═H.

5. The compound according to claim 3, wherein:
the substituent R¹ has the formula (II-A) wherein n and m are 0, X is CH₂, Y is NH and Ra═Rb=H or Ra and Rb form, together with the heteroatoms to which they linked, an imidazole or imidazoline or benzimidazole or tetrahydropyrimidine moiety, that can be unsubstituted or substituted;
the substituent R is a group —CH₂CH₂Cl.

6. The compound according to claim 4, having formula: 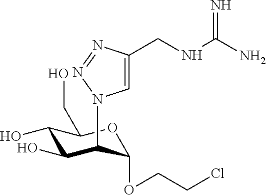
or a salt thereof.

7. The compound according to claim 3, wherein:
n 10, X═NH, m=0, Y═NH,
Rₐ═H and/or R_{b}═H.

8. The compound according to claim 1, wherein R¹ has formula (II-B): 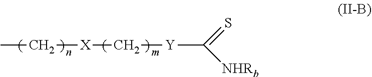
where when n=0, X is CH₂.

9. The compound according to claim 1, wherein R¹ has formula (II-C): 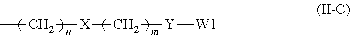
where when n=0, X is CH₂.

10. The compound according to claim 9, wherein:
X═CH₂, Y═NH,
n=0 and/or m=0.

11. The compound (I) or (IA) according to claim 1, or a salt thereof, for medical use.

12. The compound (I) or (IA) according to claim 11, or a salt thereof, wherein said compound acts as a selective inhibitor of the L-SIGN receptor.

13. The compound (I) or (IA) according to claim 11, or a salt thereof, for medical use in the prevention and/or treatment of virus infections.

14. The compound (I) or (IA) according to claim 13, or a salt thereof, wherein said virus is a virus of the Orthocoronavirinae subfamily.

15. The compound (I) or (IA) according to claim 14, or a salt thereof, wherein said virus is the SARS-CoV2 virus or a variant thereof.

16. The compound (I) or (IA) according to claim 13, or a salt thereof, wherein said virus is West Nile virus (WNV), or hepatitis C virus (HCV), or Zika virus (Zikv), or Ebola virus.

17. The compound (I) or (IA) according to claim 11, or a salt thereof, for medical use in the immunotherapy of liver tumors and/or in the treatment of immune diseases, in which said compound acts as a targeting agent to liver sinusoidal endothelial cells (L-SEC) that express the L-SIGN receptor.

18. The compound according to claim 5, wherein Ra and Rb form, together with the heteroatoms to which they linked, an imidazole or imidazoline or benzimidazole or tetrahydropyrimidine moiety, that is substituted with a substituent selected from alkyl, alkoxy, halogen, cycloalkyl, cycloalkoxy, aryl and heteroaryl.

19. The compound according to claim 6, wherein the salt has formula:

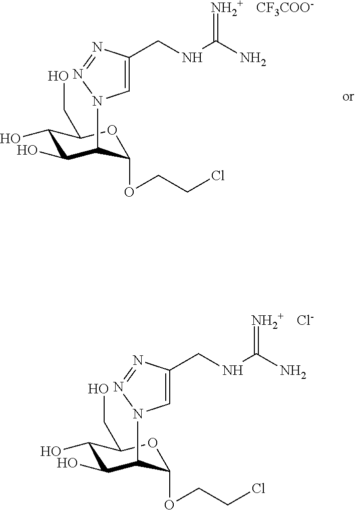

20. The compound according to claim 8, wherein:
n=0, X═CH₂, m=0, Y═NH, R_{b}═H.
